SEQUENCE LISTING (26 sequences)

SEQ ID NO: 1 - protein, 8 aa, synthetic construct
CLNSSQPS

SEQ ID NO: 2 - protein, 8 aa, synthetic construct
LNSSQPSC

SEQ ID NO: 3 - protein, 9 aa, synthetic construct
CSAYLDSLN

SEQ ID NO: 4 - protein, 9 aa, synthetic construct
SAYLDSLNC

SEQ ID NO: 5 - protein, 10 aa, synthetic construct
CSGNSTSNAC

SEQ ID NO: 6 - protein, 9 aa, synthetic construct
SGNSTSNAC

SEQ ID NO: 7 - protein, 9 aa, synthetic construct
CSGSMIAMP

SEQ ID NO: 8 - protein, 9 aa, synthetic construct
SGSMIAMPC

SEQ ID NO: 9 - protein, 9 aa, synthetic construct
CSPLSFQAT

SEQ ID NO: 10 - protein, 9 aa, synthetic construct
SPLSFQATC

SEQ ID NO: 11 - protein, 9 aa, synthetic construct
CSGSSFTMI

SEQ ID NO: 12 - protein, 9 aa, synthetic construct
SGSSFTMIC

SEQ ID NO: 13 - protein, 9 aa, synthetic construct
CSPNTNTLA

SEQ ID NO: 14 - protein, 9 aa, synthetic construct
SPNTNTLAC

SEQ ID NO: 15 - protein, 9 aa, synthetic construct
CSVDDYFTR

SEQ ID NO: 16 - protein, 9 aa, synthetic construct
SVDDYFTRC

SEQ ID NO: 17 - protein, 23 aa, synthetic construct
KAFAKLAARLYRKALARQLGVAA

SEQ ID NO: 18 - DNA, 814 bp, synthetic construct
  source 1..814: mol_type = other DNA
gatatcgctagagtattaatacgactcactataaggctagcgacatttgcttctgacacaactgtgttcactagcaacctcaaacagacaccatgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagggatcttaagctcgctttcttgctgtccaatttctattaaaggttcctttgttccctaagtccaactactaaactgggggatattatgaagggccttgagcatctggattctgcctaataaaaaccagctaacaatacactgccatcactgagctacagtgcttcatctcacgataccagctaacaatacactgccaacgcgttgagctacagtgcttcatctcatcacaccagctaacaatacactgccatcactgagctacagtgcttcatctcacgataccagctaacaatacactgccaacgcgttgagctacagtgcttcatctcaaacatttattttcattgcaatctagagcggccgc

SEQ ID NO: 19 - DNA, 856 bp, synthetic construct
  source 1..856: mol_type = other DNA
gctagagtattaatacgactcactataaggctagcgacatttgcttctgacacaactgtgttcactagcaacctcaaacagacacccaccggtcaccagctaacaatacactgccatcactgagctacagtgcttcatctcacaccggtcgacaccatgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagggatcttaagctcgctttcttgctgtccaatttctattaaaggttcctttgttccctaagtccaactactaaactgggggatattatgaagggccttgagcatctggattctgcctaataaaaaccagctaacaatacactgccatcactgagctacagtgcttcatctcacgataccagctaacaatacactgccaacgcgttgagctacagtgcttcatctcatcacaccagctaacaatacactgccatcactgagctacagtgcttcatctcacgataccagctaacaatacactgccaacgcgttgagctacagtgcttcatctcaaacatttatttt

SEQ ID NO: 20 - DNA, 856 bp, synthetic construct
  source 1..856: mol_type = other DNA
gctagagtattaatacgactcactataaggctagcgacatttgcttctgacacaactgtgttcactagcaacctcaaacagacacccaccggtcacaggccatctgtgttatatttcaccacaaggaaaaaaagccaacaccggtcgacaccatgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagggatcttaagctcgctttcttgctgtccaatttctattaaaggttcctttgttccctaagtccaactactaaactgggggatattatgaagggccttgagcatctggattctgcctaataaaaacaggccatctgtgttatatttcaccacaaggaaaaaaagccaacgatacaggccatctgtgttatattacgcgtcacaaggaaaaaaagccaatcacacaggccatctgtgttatatttcaccacaaggaaaaaaagccaacgatacaggccatctgtgttatattacgcgtcacaaggaaaaaaagccaaaacatttattttcattgcaatctagagcggcc

SEQ ID NO: 21 - protein, 121 aa, synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKGS

SEQ ID NO: 22 - DNA, 22 bp, synthetic construct
  source 1..22: mol_type = other DNA
accagctaacaatacactgcca

SEQ ID NO: 23 - DNA, 22 bp, synthetic construct
  source 1..22: mol_type = other DNA
tgagctacagtgcttcatctca

SEQ ID NO: 24 - DNA, 21 bp, synthetic construct
  source 1..21: mol_type = other DNA
acaggccatctgtgttatatt

SEQ ID NO: 25 - DNA, 19 bp, synthetic construct
  source 1..19: mol_type = other DNA
cacaaggaaaaaaagccaa

SEQ ID NO: 26 - RNA, 29 nt, synthetic construct
  source 1..29: mol_type = other RNA
ggggcgtgatccgaaaggtgacccggatc

(19) United States
(12) Patent Application Publication
(10) Pub. No.: US 2026/0306665 A1
(43) Pub. Date: Oct. 8, 2026

(54) TARGETED LIPID NANOPARTICLES FOR TREATING CENTRAL NERVOUS SYSTEM INJURIES

(71) Applicant: Wisconsin Alumni Research Foundation, Madison, WI (US)

(72) Inventors: Daniel Hellenbrand, Lodi, WI (US); Jacobus Burger, Madison, WI (US); William Murphy, Waunakee, WI (US); Amgad Hanna, Waunakee, WI (US)

(21) Appl. No.: 19/641,651

(22) Filed: Apr. 8, 2026

Related U.S. Application Data
(60) Provisional application No. 63/785,507, filed on Apr. 8, 2025.

(51) Int. Cl. A61K 48/00; A61K 9/1272; A61K 31/7105; A61K 38/20; A61K 47/64; A61K 47/69; A61P 25/28

(52) U.S. Cl. CPC A61K 48/0033; A61K 9/1272; A61K 31/7105; A61K 38/2013; A61K 38/2026; A61K 38/2066; A61K 38/2086; A61K 47/64; A61K 47/6911; A61P 25/28

(57) ABSTRACT

The present invention provides lipid nanoparticles that comprise a therapeutic agent and have an astrocyte targeting peptide linked to their surface. Suppressor RNAs that restrict transgene expression to the central nervous system or to astrocytes, in particular, are also provided. Methods of using the lipid nanoparticles and suppressor RNAs in combination of therapeutic mRNAs to treat a central nervous system injury in a subject are also provided.

### CROSS-REFERENCE TO RELATED APPLICATIONS

This patent application claims the benefit of U.S. Provisional Application No. 63/785,507 filed on Apr. 8, 2025, the entire contents of which are incorporated herein by reference.

### STATEMENT REGARDING FEDERALLY SPONSORED RESEARCH

This invention was made with government support under NS136564 awarded by the National Institutes of Health. The government has certain rights in the invention.

### SEQUENCE LISTING STATEMENT

A Sequence Listing accompanies this application and is submitted as an XML file of the sequence listing named “96029604757_SL.xml” which is 26,303 bytes in size and was created on Apr. 7, 2026. The sequence listing is electronically submitted via Patent Center with the application and is incorporated herein by reference in its entirety.

### BACKGROUND

Central nervous system (CNS) injuries, such as spinal cord injury (SCI) and traumatic brain injury (TBI), are severe life altering traumas that leave patients with major functional deficits. Spinal cord injury (SCI) results in sensory, motor, and autonomic impairments along with other associated medical conditions that severely decrease the patient's quality of life. According to the National Spinal Cord Injury Statistical Center (NSCISC), there are approximately 305,000 patients living with SCI in the United States with approximately 18,000 new SCI cases each year.¹ Due to the loss of function, SCI patients are often unable to care for themselves and unable to maintain employment, which places an enormous expense on both the patient and the government.¹ However, despite the considerable economic and societal challenges, no therapy is available for substantial recovery of functional deficits after SCI. Accordingly, there is a continued need for methods that retain and/or restore function following spinal cord injury (SCI).

After SCI, extensive inflammation occurs, which has been shown to lead to additional neural damage. However, SCI patients are more susceptible to various types of infections, making systemic immunosuppression an impractical treatment strategy. Immunosuppressive regulatory T cells (T_{regs}) are potent cells capable of producing multiple anti-inflammatory agents and reducing neuroinflammation. Although T_{regs} possess several beneficial attributes, their numbers are low after SCI. Interleukin-2 (IL-2), a cytokine that promotes proliferation and survival of T_{regs}, is also significantly decreased after SCI. Thus, local upregulation of IL-2 is a promising means for limiting the inflammation-induced damage caused by SCI.

Within days post-injury, microglia, which proliferate and transform into amoeboid phagocytic cells, and peripherally derived macrophages (PDMs) make up the majority of the inflammatory cells present at the lesion site. Anti-inflammatory cytokines (e.g., IL-4, IL-10, IL-13) can shift macrophages to an anti-inflammatory state, reducing the release of pro-inflammatory cytokines, and thereby reducing the exacerbated amount of infiltrating immune cells.

IL-10 is a potent anti-inflammatory cytokine that has been shown to downregulate the synthesis of several pro-inflammatory species and pro-apoptotic factors. Moreover, exposing infiltrating monocytes to IL-10 has been shown to shift them to a beneficial IL-10 producing state. IL-4 and IL-13 are related anti-inflammatory cytokines that regulate many aspects of inflammation and have been shown to shift macrophages to an anti-inflammatory state. Both IL-4 and IL-10 exert neuroprotective effects against microglia/macrophage mediated neuronal toxicity. Additionally, cell-based delivery of IL-13 increased the number of alternatively activated macrophages and improved functional outcome after SCI in mice. These data demonstrate the capability of anti-inflammatory cytokines to serve as a therapeutic intervention after SCI.

### SUMMARY

In a first aspect, the present invention provides lipid nanoparticles comprising a therapeutic agent and an astrocyte targeting peptide, wherein the astrocyte targeting peptide is on the surface of the lipid nanoparticle.

In a second aspect, the present disclosure provides suppressor messenger RNAs (mRNAs) and therapeutic mRNAs that may be used together to allow for expression of a therapeutic protein encoded by the therapeutic mRNA in the central nervous system (CNS). The suppressor mRNA and the therapeutic mRNA may be encoded by one or more constructs in which the nucleotide sequence encoding the mRNAs is operably connected to a promoter. The suppressor mRNA includes a nucleotide sequence encoding a translational repressor protein, such as L7Ae, and comprises one or more astrocyte-expressed microRNA (miRNA) target sites in the 5′ untranslated region (UTR) and/or 3′ UTR. The therapeutic mRNA comprises a repressor binding domain and a nucleotide sequence encoding a therapeutic protein or reporter protein.

In a third aspect, the present disclosure provides methods of treating a central nervous system (CNS) injury in a subject. The methods comprise administering a lipid nanoparticle to the subject that is delivered to astrocytes surrounding the CNS injury.

### BRIEF DESCRIPTION OF THE DRAWINGS

FIG. 1 shows that IL-2 is downregulated after rat SCI. A 10-gram weight was dropped from varying heights to induce SCI. More severe injuries result in earlier and significantly lower levels of IL-2 compared to mild injuries.¹⁵

FIG. 2 shows that IL-4, IL-10, and IL-13 are downregulated after rat SCI. A 10-gram weight was dropped from varying heights to induce SCI. Local levels of IL-4, IL-10, and IL-13 all significantly decreased within one week after SCI.¹⁵

FIG. 3 shows a strategy for developing highly efficient, astrocyte-targeting LNPs for IV administration after SCI.

FIG. 4 shows higher translation of reporter genes from LNPs with astrocyte specific peptides. Rat astrocyte cell cultures transfected with LNPs delivering eGFP mRNA (A). Specific surface peptides on LNPs increased luciferase translation in rat astrocytes (B) and in astrocytes derived from human induced pluripotent stem cells (iPSCs) (C).

FIG. 5 shows that IL-6 was reduced when the KAFAK peptide was added to LPS activated astrocytes (A). Adding the KAFAK peptide to the LNP surface significantly increased astrocyte transfection compared to standard uncoated LNPs (B).

FIG. 6 shows that, when delivered with the reporter mRNA, the mRNA repressor L7Ae is translated and blocks the reporter gene from being translated (A). There was excellent eGFP transfection in rat astrocytes without L7Ae (B), which was almost completely blocked with the addition of the L7Ae mRNA (C&D). The sham mRNA suppressor was inefficient at blocking transfection, demonstrating that the effects are specific to L7Ae (D). Four repeats of each of two astrocyte-expressed miRNA target sites were added to the 3′ UTR of luciferase mRNA (E). There was efficient astrocyte transfection from the control mRNA and almost no transfection when the luciferase mRNA containing the miRNA sequences was used (F), demonstrating that the selected miRNAs are present in astrocytes and that luciferase mRNA is degraded before translation. The large difference in astrocyte transfection between control mRNA and the 4×2T mRNA was not observed in neuron cell cultures (G), demonstrating that the selected miRNAs are not present in neurons. When we include these miRNA target sites in both the 3′ and 5′ UTRs of L7Ae (H-I), the L7Ae repressor mRNA is degraded in astrocyte cultures, allowing for translation of the reporter gene (I), which was significantly higher than in neurons (J).

FIG. 7 shows the distribution of LNP-delivered transgene expression. Rats were subjected to a T10 SCI, and standard uncoated firefly luciferase (Fluc) mRNA-loaded LNPs were injected via a jugular vein catheter (JVC) 30 minutes post-injury. The spinal cords and organs were harvested 24 hours post-injury and incubated ex vivo in 10 mg/ml luciferin for 15 min prior to imaging. In uninjured rats, the spleen was transfected, but no transfection occurred in the spinal cord (A). Rats with SCI had a disrupted blood-spinal cord barrier (BSCB) and exhibited over a 60-fold increase in relative light units (RLU) compared to untreated (UT) rats that had a SCI and no LNP treatment (A-B). With standard LNPs, the spleen and liver are transfected (C), highlighting the benefit of including the L7Ae repressor circuit.

FIG. 8 shows that LNPs loaded with IL-2 mRNA can be used to increase IL-2 levels in the spinal cord. Rats were subjected to a T10 SCI, and standard uncoated Fluc mRNA-loaded LNPs for IHC (A), vHH mRNA-loaded LNPs for flow cytometry (B-C), and IL-2 mRNA-loaded LNPs (D) were injected via a JVC 30 minutes post-injury and the spinal cords were harvested 24 hours post-injury. Transfected astrocytes and microglia are shown on transverse sections, white arrows=transfected cells and red arrows=cells not transfected (A). After harvesting ~7 mm of spinal cord centered on the injury, removing myelin debris with beads, and creating a single-cell suspension, flow cytometry revealed that nearly 70% of the cells were transfected (B). There were distinct groupings of specific cells (C). Although there were less Iba1+ microglia cells (3.62% of total cells), they were highly transfected (C). ACSA+ Astrocytes were prevalent (40.6% of total cells) and most were transfected (C). A group of transfected cells (ACSA-&Iba1-), likely neutrophils based on their smaller size and the 24-hour post-injury timeline, was identified. LNPs loaded with IL-2 mRNA significantly increased IL-2 levels in the spinal cord compared to injured controls 24 hours post-SCI (D).

### DETAILED DESCRIPTION

The present invention provides lipid nanoparticles that comprise a therapeutic agent and have an astrocyte targeting peptide linked to their surface. Suppressor RNAs that restrict transgene expression to the central nervous system and in particular to astrocytes and methods of using the lipid nanoparticles and suppressor RNAs to treat a central nervous system injury in a subject are also provided.

The present inventors have developed a novel drug delivery platform that specifically targets central nervous system (CNS) injuries. In this platform, lipid nanoparticles comprising an astrocyte targeting peptide are used to deliver a therapeutic agent. In the Examples, the inventors demonstrate that, when these lipid nanoparticles are delivered intravenously, they enter CNS injury sites through damaged blood vessels. Thus, these lipid nanoparticles offer an innovative way to target therapeutics to any CNS injury in which the vasculature is disrupted. The inventors further demonstrate that the RNA repressor L7Ae:K-turn system can be used to restrict transgene expression to astrocytes, suggesting that this system can be included in the lipid nanoparticles to increase the specificity of their therapeutic effect. Finally, the inventors demonstrate that their lipid nanoparticles can be used to increase IL-2 levels at sites of spinal cord injury via delivery of mRNA encoding IL-2. Thus, these nanoparticles are a promising means to reduce local inflammation and thereby improve outcomes following CNS injury.

After CNS injury, there is immediate mechanical damage causing cell death and breakdown of blood vasculature, which sets off a secondary injury cascade that continues for months, leading to additional cell death and CNS damage. Implementing therapies that reduce this secondary damage within the early acute phase is challenging due to timing and location. Administering a local injection to the injury site requires imaging to confirm correct injection location and to ensure healthy CNS tissue is not damaged during injection, and there is typically not enough time within the therapeutic time window to perform this imaging. Although administering a systemic therapy is faster, such therapies can cause unwanted off-target side effects. The lipid nanoparticles described herein are specifically designed to overcome the aforementioned challenges.

### Lipid Nanoparticles

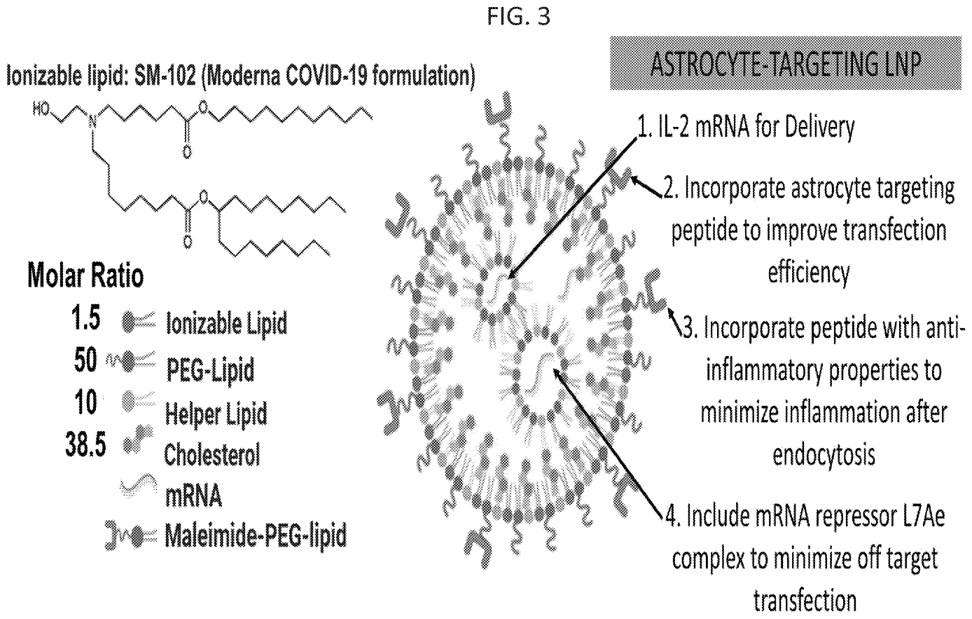

In a first aspect, the present invention provides lipid nanoparticles comprising a therapeutic agent and an astrocyte targeting peptide, wherein the astrocyte targeting peptide is on the surface of the lipid nanoparticle. As used herein, a “lipid nanoparticle” (abbreviated LNP) is a spherical vesicle made of lipids. Lipid nanoparticles may range in size from 0.2 nm to 1000 nm, from 1 nm to 500 nm, from 5 nm to 300 nm, or from 10 nm to 200 nm. In some embodiments, the LNP has a size of ~140 nm±20 nm. A lipid nanoparticle can comprise a variety of different types of lipids. For example, a lipid nanoparticle may include ionizable lipids, structural lipids, sterols, pegylated lipids, as shown in FIG. 3. The pegylated lipids used in the lipid nanoparticles may be functionalized. For example, the pegylated lipids may be chemically functionalized pegylated lipids or antibody-functionalized pegylated lipids.

An “ionizable lipid” is an amphiphilic lipid that can change its charge depending on the pH of the environment. The inclusion of ionizable lipids in LNPs allows the LNPs to efficiently encapsulate nucleic acids and aids in endosomal escape. In the Examples, the inventors used the ionizable lipid SM-102 in their LNPs. Thus, in some embodiments, the ionizable lipid is SM-102. Other exemplary ionizable lipids include Dlin-MC3-DMA and ALC-0315. In some embodiments, the ionizable lipids make up between 20 and 80 mol %, between 30 and 70 mol %, between 40 and 60 mol %, or about 50 mol % of the lipids in the lipid nanoparticle. A mole percent (mol %) expresses the concentration of a component in a mixture. Mole percent is calculated by dividing the number of moles of the component by the total number of moles in the mixture, then multiplying by 100 to get a percentage. Herein, the components may refer to lipids and the mixture may refer to an LNP.

A “structural lipid” is a lipid used to form and maintain the structure of the LNP. Structural lipids can be used to improve the stability of LNPs and assist in encapsulation/endosomal release of the therapeutic agent. In the Examples, the inventors used the structural lipid 1,2-distearoyl-sn-glycero-3-PC (1,2-DSPC) in their LNPs. Thus, in some embodiments, the structural lipid is 1,2-DSPC. Other exemplary structural lipids include 1,2-dioleoyl-3-trimethylammonium propane (DOTAP) and 1,2-Dioleoyl-sn-glycero-3-phosphoethanolamine (DOPE). In some embodiments, the structural lipids make up between 1 and 20 mol %, between 5 and 15 mol %, or about 10 mol % of the lipids in the lipid nanoparticle.

“Sterols” are a subgroup of steroids with a hydroxyl group at the 3-position of the A-ring. Sterols may be used to provide stability, flexibility, and membrane structure to LNPs in addition to aiding endosomal escape. In the Examples, the inventors used the sterol cholesterol in their LNPs. Thus, in some embodiments, the sterol is cholesterol. In some embodiments, the sterol is sitosterol. In some embodiments, the sterols make up between 10 and 70 mol %, between 20 and 60 mol %, between 30 and 50 mol %, or about 35.8 mol % of the lipids in the lipid nanoparticle.

A “pegylated lipid” is a lipid to which polyethylene glycol (PEG) has been attached. PEG is a hydrophilic polymer. PEGylation may enhance the LNP's stability, solubility, and bioavailability, while also reducing immunogenicity. In the Examples, the inventors utilized the pegylated lipids 1,2-dimyristoyl-rac-glycero-3-methoxypolyethylene glycol-2000 (DMG-PEG2000) and 1,2-distearoyl-sn-glycero-3-phosphoethanolamine-N-[maleimide(polyethylene glycol)-2000] in their LNPs. Thus, in some embodiments the LNPs comprise one or both of these specific pegylated lipids. In some embodiments, the LNPs comprise the pegylated lipid 1,2-distearoyl-sn-glycero-3-phosphoethanolamine-N—[PDP(polyethylene glycol)-2000]. In some embodiments, the pegylated lipids make up between 0.1 and 10.0 mol %, between 0.5 and 5.0 mol %, between 1.0 and 2.0 mol %, or about 1.5 mol % of the lipids in the lipid nanoparticle. In some embodiments, DMG-PEG2000 makes up between 0.1 and 10 mol %, between 0.5 and 5 mol %, between 1.0 and 2.0 mol %, or about 1.3 mol % of the lipids in the lipid nanoparticle. In some embodiments, 1,2-distearoyl-sn-glycero-3-phosphoethanolamine-N-[maleimide(polyethylene glycol)-2000] makes up between 0.1 and 5 mol %, between 0.2 and 3 mol %, between 0.2 and 1.0 mol %, or about 0.2 mol % of the lipids in the lipid nanoparticle.

In the Examples, the inventors made LNPs comprising an ionizable lipid, a structural lipid, a sterol, and a pegylated lipid at a molar ratio of 50:10:38.5:1.5. Thus, in preferred embodiments, the molar ratio of the ionizable lipid, the structural lipid, the sterol, and the pegylated lipid is 50:10:38.5:1.5.

Astrocytes are a type of glial cell found in the CNS, specifically in the brain and spinal cord. They are star-shaped cells that provide structural and metabolic support to neurons. The term “astrocyte targeting peptide” is used herein to describe a peptide that specifically binds to a moiety that is found on the surface of astrocytes and is not found, or is less prevalent, on other cell types. Thus, inclusion of an astrocyte targeting peptide on the surface of an LNP helps to target the LNP to astrocytes. In the Examples, the inventors demonstrated that adding an astrocyte targeting peptides selected from SEQ ID NO: 1 (AS1-N), SEQ ID NO: 2 (AS1-C), SEQ ID NO: 5 (A-21-1-N), SEQ ID NO: 7 (A+C1-11-N), SEQ ID NO: 8 (A+C1-11-C), SEQ ID NO: 9 (A+C4-9-N), SEQ ID NO: 10 (A+C4-9-C), SEQ ID NO: 12 (ADC6-5-C), SEQ ID NO: 13 (AC2-6-N), and SEQ ID NO: 16 (A+C5-1-C) to their LNPs increases transfection into astrocytes, and that the astrocyte targeting peptides of SEQ ID NO: 10 (A+C4-9-C), SEQ ID NO: 12 (ADC6-5-C), or SEQ ID NO: 13 (AC2-6-N) significantly increase transfection into astrocytes. Thus, in some embodiments, the astrocyte targeting peptide is selected from one of these tested peptides.

The lipid nanoparticle may further comprise an anti-inflammatory peptide. As used herein, the term “anti-inflammatory peptide” refers to short chain of amino acids (e.g., 2-50 amino acids) that has the ability to modulate or reduce inflammation in a subject. Anti-inflammatory peptides may target proteins, enzymes, or receptors involved in the inflammatory process, helping to downregulate or counteract excessive inflammatory responses. In the Examples, the inventors generated LNPs comprising the anti-inflammatory peptide KAFAK (SEQ ID NO: 17). Thus, in some embodiments, the anti-inflammatory peptide is KAFAK. In embodiments in which the lipid nanoparticles comprise an anti-inflammatory peptide, any molar ratio of anti-inflammatory peptide to the astrocyte targeting peptide may be utilized. This molar ratio can be varied based on the desired effects or specific experimental conditions. In some embodiments, this molar ratio is between 0.1:1 and 1:1.

Both the astrocyte targeting peptide and the anti-inflammatory peptide may be linked to the lipid nanoparticle surface via a pegylated lipid. In the Examples, this was accomplished by linking a thiol group on the peptide (via a terminal cysteine residue) to a maleimide group on a pegylated lipid via a thiosuccinimide bond. Thus, in some embodiments, the astrocyte targeting peptide and/or the anti-inflammatory peptide comprises a thiol group and the pegylated lipid comprises a maleimide group. In other embodiments, the astrocyte targeting peptide and the anti-inflammatory peptide are linked to the lipid nanoparticle surface via thiol exchange with a pyridyldithiol group (PDP).

The LNPs of the present invention comprise a therapeutic agent. As used herein, the term “therapeutic agent” refers to any substance that aids in the treatment, prevention, or diagnosis of a disease or condition. Examples of therapeutic agents include, but are not limited to, pharmaceuticals, biologics, toxins, alkylating agents, enzymes, antibiotics, antimetabolites, antiproliferative agents, chemotherapeutic agents, hormones, neurotransmitters, polynucleotides, aptamers, lectins, compounds that alter cell membrane permeability, photochemical compounds, small molecules, liposomes, micelles, peptides, gene therapy vectors, and vaccines. In some embodiments, the therapeutic agent is a therapeutic RNA or a therapeutic protein. In some specific embodiments, the therapeutic agent is an mRNA that encodes IL-2, IL-4, IL-10, or IL-13.

Suppressor mRNA

In a second aspect, the present disclosure provides suppressor messenger RNAs (mRNAs) that comprise a nucleotide sequence encoding a translational repressor protein, such as L7Ae, and also comprise one or more astrocyte-expressed microRNA (miRNA) target sites in the 5′ untranslated region (UTR) and/or 3′ UTR. The suppressor mRNAs are designed to prevent protein expression from a therapeutic or reporter mRNA in cells types other than astrocytes. The mRNA encoding the therapeutic or reporter protein (the “therapeutic mRNA”) comprises a repressor protein binding domain, such as a K-turn, to which the repressor protein binds and prevents translation of the therapeutic or reporter protein. In the presence of the astrocyte-expressed miRNA, the miRNA binds to the astrocyte-expressed miRNA target site in the suppressor mRNA and cleaves the mRNA such that the repressor protein is not translated and this allows the therapeutic mRNA to be translated and generate the therapeutic or reporter protein only in cells expressing the miRNA.

In other words, the paired suppressor mRNA and therapeutic mRNA are designed to restrict expression of the therapeutic or reporter protein to astrocytes. In astrocytes, astrocyte-expressed miRNAs (i.e., miRNAs that are found in astrocytes) bind to the astrocyte-expressed miRNA target site(s) in the suppressor mRNA, which results in its degradation and allows for expression of the therapeutic mRNA. In other cell types, the astrocyte-expressed miRNAs are not present or present at decreased levels, so the suppressor mRNA is not degraded and the archaeal protein L7Ae (SEQ ID NO: 21) is expressed from it. L7Ae binds to a specific RNA structural motif called a “K-turn” and blocks ribosomal translation. Thus, including a K-turn in the therapeutic mRNA and co-delivering the suppressor mRNA to cells along with the therapeutic mRNA restricts protein expression from the therapeutic mRNA to astrocytes.

As used herein, an “astrocyte-expressed miRNA target site” is a sequence that is complementary to an astrocyte-expressed miRNA. The term astrocyte-expressed miRNA refers to a miRNA that is expressed within astrocytes. An astrocyte-expressed miRNA may also be expressed in cell types other than astrocytes. However, in some embodiments, the astrocyte-expressed miRNA is an astrocyte-specific miRNA that is found in astrocytes but not in other cell types. The term “complementary” refers to the ability of a nucleic acid molecule to bind to (i.e., hybridize with) another nucleic acid molecule through the formation of hydrogen bonds between specific nucleotides (i.e., A with T or U and G with C), forming a double-stranded molecule. The astrocyte-expressed miRNA target site(s) used with the present invention may be complementary to only a portion of an astrocyte-expressed miRNA and may be imperfectly complementary (e.g., contain mismatches), so long as they bind to the astrocyte-expressed miRNA specifically (i.e., they bind to the astrocyte-expressed miRNA in preference to other polynucleotides) and allow for expression of the mRNA in astrocytes. However, in the Examples, the inventors utilized astrocyte-expressed miRNA target sites that are completely complementary to astrocyte-expressed miRNAs. Thus, in some embodiments, the astrocyte-expressed miRNA target site(s) are 100% complementary to astrocyte-expressed miRNA(s).

As is described in the Examples, the inventors generated suppressor mRNAs that comprise target sites for two astrocyte-expressed miRNAs, namely miR-449a and miR-143. While one miRNA target site is sufficient to induce gene suppression, the inventors utilized two different miRNA target sites to increase their chances of selecting a miRNA sufficient for degradation to allow for expression in astrocytes. Thus, in some embodiments, the suppressor mRNAs comprise at least one copy of a first astrocyte-expressed miRNA target site and at least one copy of a second astrocyte-expressed miRNA target site. In some embodiments, the first astrocyte-expressed miRNA target site is complementary to miR-449a and the second astrocyte-expressed miRNA target site is complementary to miR-143. For example, in some embodiments, the first astrocyte-expressed miRNA target site comprises SEQ ID NO: 22 and the second astrocyte-expressed miRNA target site comprises SEQ ID NO: 23.

In other embodiments, the suppressor mRNAs comprise target sites for the astrocyte-expressed miRNAs miR-410 and miR-135a. In these embodiments, the suppressor mRNAs comprise one or more astrocyte-expressed miRNA target sites that are complementary to miR-410 and miR-135a. For example, in some embodiments, the first astrocyte-expressed miRNA target site comprises SEQ ID NO: 24 and the second astrocyte-expressed miRNA target site comprises SEQ ID NO: 25.

Two different suppressor mRNAs were tested in the Examples, including one suppressor mRNA that had four copies of the miR-449a target site and four copies of the miR-143 target site in the 3′ UTR (miR-449a_miR-143-bg-L7Ae-bg-4×2T; SEQ ID NO: 18), and another suppressor mRNA that had four copies of the miR-449a target site and four copies of the miR-143 target site in the 3′ UTR as well as one copy of the miR-449a target site and one copy of the miR-143 target site in the 5′ UTR (miR-449a_miR-143-bg-2T-L7Ae-4×2T; SEQ ID NO: 19). Thus, in some embodiments, the suppressor mRNAs comprise at least four copies of the first astrocyte-expressed miRNA target site and at least four copies of the second astrocyte-expressed miRNA target site in the 3′ UTR. In some embodiments, the suppressor mRNAs further comprise at least one copy of the first astrocyte-expressed miRNA target site and at least one copy of the second astrocyte-expressed miRNA target site in the 5′ UTR. In some embodiments, the suppressor mRNA comprises SEQ ID NO: 18 or SEQ ID NO: 19. The inventors have also generated another suppressor mRNA that includes four copies of the miR-410 target site and four copies of the miR-135a target site in the 3′ UTR as well as one copy of the miR-410 target site and one copy of the miR-135a target site in the 5′ UTR (miR-410_miR-135a-bg-2T-L7Ae-4×2T; SEQ ID NO: 20). Thus, in some embodiments, the suppressor mRNA comprises SEQ ID NO: 20. Those of skill in the art will understand that these miRNAs can be mixed and matched with each other and include multiple copies of the target to ensure that any secondary structure within the mRNA still allows for the miRNA to mark the suppressor RNA for degradation and block translation of the repressor protein.

Also provided are constructs capable of encoding the suppressor and therapeutic mRNAs. The constructs comprise a nucleotide sequence encoding the mRNAs operably linked to promoters to allow the expression of the mRNAs. The promoter may be any suitable promoter, and the two mRNAs may be encoded on a single construct or two separate constructs. If a single construct is used the two mRNA may share a single promoter and be separated by an IRES to allow for separate translation of the two mRNAs. The two mRNAs may be generated separately and combined for delivery in any ratio. The ratio may be 1:1 of suppressor mRNA to therapeutic mRNA, but better expression was observed when the amount of suppressor mRNA to therapeutic mRNA was lower such as 0.1 to 1, 0.2:1, 0.5:1, 0.75:1 or even 1:1. The constructs for use in this invention may be plasmids, viral vectors, or any other genetic constructs available to those of skill in the art.

Lipid nanoparticles that comprise the suppressor mRNAs and/or therapeutic mRNAs described herein are also provided. In some embodiments, these lipid nanoparticles comprise a therapeutic mRNA encoding a therapeutic protein that comprises a K-turn or other repressor protein binding site in its 5′ UTR. In some embodiments, the K-turn comprises SEQ ID NO: 26. Therapeutic proteins and reporter proteins for use in the therapeutic mRNA may include but are not limited to cytokines such as IL-2, IL-4, IL-10, IL-13 or a reporter protein such as GFP, YFP, luciferase or any other reporter protein.

### Methods of Treatment

In a third aspect, the present disclosure provides methods of treating a CNS injury in a subject. The methods comprise administering a lipid nanoparticle to the subject that is delivered to astrocytes surrounding the CNS injury.

As used herein, the term “central nervous systems injury” refers to any injury that causes a break in vasculature within the CNS. In some embodiments, the CNS injury is a spinal cord injury, a traumatic brain injury, hypertensive encephalopathy, vascular dementia, multiple sclerosis, an aneurysm, or a hemorrhagic stroke. The lipid nanoparticle administered to the subject in these methods may be any of the lipid nanoparticles described herein.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraoral administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be performed in multiple doses and may be continuous or intermittent. In preferred embodiments, the lipid nanoparticle is administered to the subject intravenously. In other embodiments, the lipid nanoparticle is administered to the subject via intracranial injection or intraspinal injection.

After CNS injury, there is immediate mechanical damage causing cell death and breakdown of blood vasculature, which sets off a secondary injury cascade that continues for months. It is thought that prompt treatment is required to prevent or reduce the secondary injury cascade. Thus, in preferred embodiments, the LNP is administered between 1 and 24 hours, between 2 and 12 hours, or between 4 and 8 hours of the CNS injury.

As used herein, “treating” describes something that is done to a subject to combat a health problem (i.e., an injury, disease, condition, or disorder). Treating may involve controlling the health problem, lessening its symptoms or complications, slowing its progression, and/or eliminating it. Here, treating may also refer to limiting the damage caused by a CNS injury or inhibition of further injury. In preferred embodiments, the lipid nanoparticle is administered in a therapeutically effective amount. The term “therapeutically effective amount” refers to an amount sufficient to produce beneficial or desirable clinical results.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

The terms “nucleic acid,” “oligonucleotide,” and “polynucleotide” are used interchangeably to refer a polymer of DNA or RNA. A nucleic acid may be single-stranded or double-stranded and may represent the sense or the antisense strand. A nucleic acid may be synthesized or obtained from a natural source. A nucleic acid may contain natural, non-natural, or altered nucleotides, as well as natural, non-natural, or altered internucleotide linkages (e.g., phosphoroamidate linkages, phosphorothioate linkages). The term nucleic acid encompasses constructs, plasmids, vectors, and the like.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

Unless defined otherwise, all technical and scientific terms used herein have the same meaning as commonly understood by one of skill in the art to which the invention pertains. All definitions, as defined and used herein, should be understood to control over dictionary definitions, definitions in documents incorporated by reference, and/or ordinary meanings of the defined terms.

The following examples are meant only to be illustrative and are not meant as limitations on the scope of the invention or of the appended claims.

### Examples

The initial insult after spinal cord injury (SCI) causes cell death and breakdown of the blood-spinal cord barrier (BSCB), triggering a secondary cascade that results in extensive immune cell infiltration, further neuronal death, and additional spinal cord damage.²⁻⁴ Although, immunosuppressive regulatory T cells (T_{regs}) are potent immunoregulatory cells capable of producing multiple anti-inflammatory agents,^{5,6} reducing neutrophil cytotoxicity,⁷⁻⁹ promoting an anti-inflammatory state in macrophages/microglia,¹⁰ and promoting remyelination,^{11, 12} T_{reg} numbers are low post-SCI.¹⁰ Additionally, IL-2, a cytokine that promotes extensive proliferation and survival of T_{regs},^{13, 14} is depleted after SCI,¹⁵ highlighting the need for exogenous mechanisms to increase IL-2 levels and, consequently, boost T_{reg} numbers.

Accordingly, as is described in the following example, the present inventors have developed lipid nanoparticles (LNPs) designed to upregulate IL-2 in astrocytes (i.e., an abundant cell type that encapsulates CNS injuries) via non-viral mRNA delivery. The LNPs are unable to cross the intact blood-brain barrier (BBB) or BSCB and thus can only transfect astrocytes within the injured CNS tissue. To enhance astrocyte endocytosis, the LNPs include astrocyte binding peptides incorporated on their surface. To minimize transfection in off-target cells, an RNA repressor L7Ae:K-turn system was developed with binding motifs for astrocyte specific miRNAs incorporated into the L7Ae RNA complex.

### Introduction

Regulatory T Cells (T_{regs}) can Reduce Inflammation after SCI

Primary insult after SCI initiates a complex pathophysiology consisting of exacerbated inflammation and culminating in a dense glial scar.²⁻⁴ Although it has been shown that the inflammatory response after SCI is beneficial in removing debris and releasing neurotrophic factors, the excessive inflammation and extensive infiltration of immune cells leads to additional neural damage. Within the first hour post-SCI, several inflammatory cytokines are secreted by microglia, astrocytes, and peripherally derived macrophages (PDMs) within the lesion.^{3, 15-18} Throughout the first few hours post-injury, neutrophils are the predominant infiltrating cells and over-activation of these cells causes tissue destruction through the release of significant amounts of neurotoxins.¹⁹⁻²¹ Within days post-injury, microglia, which proliferate and transform to amoeboid phagocytic cells, and PDMs make up the majority of inflammatory cells present within the lesion.^{17, 22-25} Although initially reactive astrocytes migrate to the lesion, proliferate extensively and aid in the tissue repair process after SCI, they eventually become scar-forming astrocytes and form a border around the lesion.²⁶ This astrocytic border encapsulating the lesion, protects the remaining intact spinal cord, and inhibits the spread of infiltrating immune cells.^{27, 28} After SCI, there is extensive glutamate release and feedback of the inflammatory response, and thus the microglia/PDMs take on an inflammatory state expressing very low levels of MHC II molecules and characterized by excessive release of IL-10, IL-6, and TNFα. This leads to toxicity causing astrocytes and neurons to show signs of damage, expressing reactive oxygen species capable of inducing apoptosis.²⁹ However, these cells can alternatively be activated toward a more anti-inflammatory state with the ability to promote tissue remodeling, neurotrophic support, and axonal regeneration.³⁰⁻³⁴ The inflammatory state of microglia/PDMs exists on a complex continuum, and is dictated by signaling cues in the extracellular environment.

T_{regs} are potent immunoregulatory cells capable of producing multiple anti-inflammatory agents and protecting against autoimmunity.³⁵ Importantly, T_{regs} are common in blood, and recently it has been demonstrated that there is a small population of resident T_{regs} in the central nervous system (CNS).^{14, 36, 37} T_{regs} secrete TGF-β capable of inducing neutrophil apoptosis^{8, 38} and promoting polymorphonuclear myeloid-derived suppressor cells (PMN-MDSCs) with immunosuppressive functions,^{9, 39} and thus reducing the inflammatory environment in acute SCI.^{7, 8, 38, 40-42} Recently, it has been demonstrated that neutrophils are a heterogenous population.^{9, 39, 43, 44} An important subset of low-density neutrophils (LDNs) are PMN-MDSCs that have a reduced oxidative burst, reduced inflammation, and reduced cytotoxicity compared to normal density neutrophils (NDNs).^{9, 39} T_{regs} also promote an anti-inflammatory state in microglia/PDMs by secreting IL-10.^{5, 45} Moreover, T_{reg} depletion after SCI induced a proinflammatory state in microglia/PDMs and an upregulation in TNFα and IL-6.¹⁰ Taken together, these prior studies clearly demonstrate that T_{regs} produce critical factors that promote an anti-inflammatory microglia/PDM state. In addition to T_{regs} immunoregulatory functions, T_{regs} possess a regenerative role by secreting CCN3, a protein which directly signals oligodendrocyte precursors to differentiate into myelin-producing cells,^{11, 12} and suppress neurotoxic reactive astrocytes by secreting amphiregulin.^{46, 47} Although T_{regs} possess several beneficial attributes, their numbers are low post-SCI,^{10, 14} demonstrating a need for mechanisms to increase T_{reg} levels.

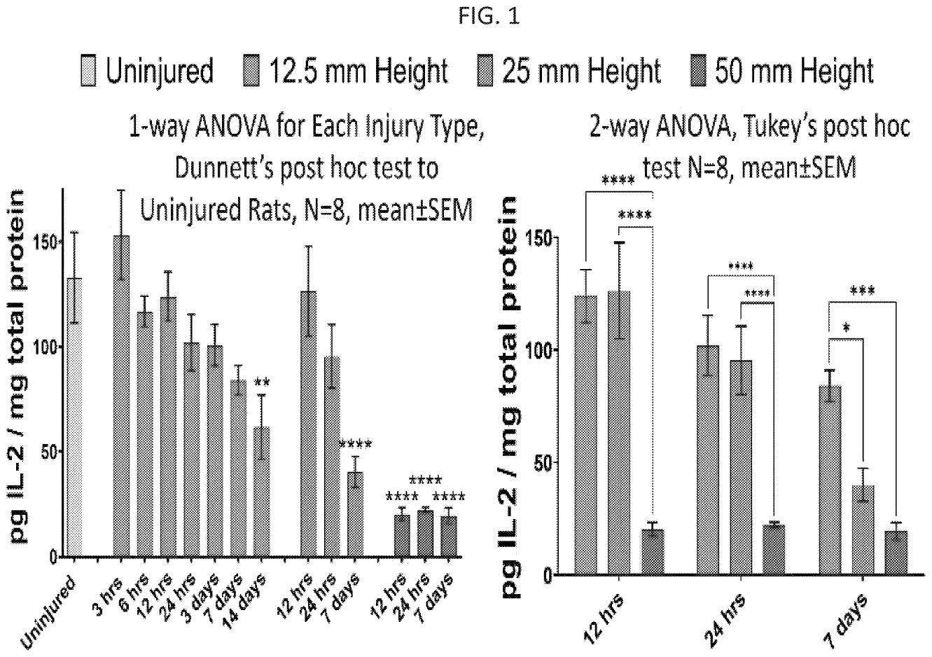

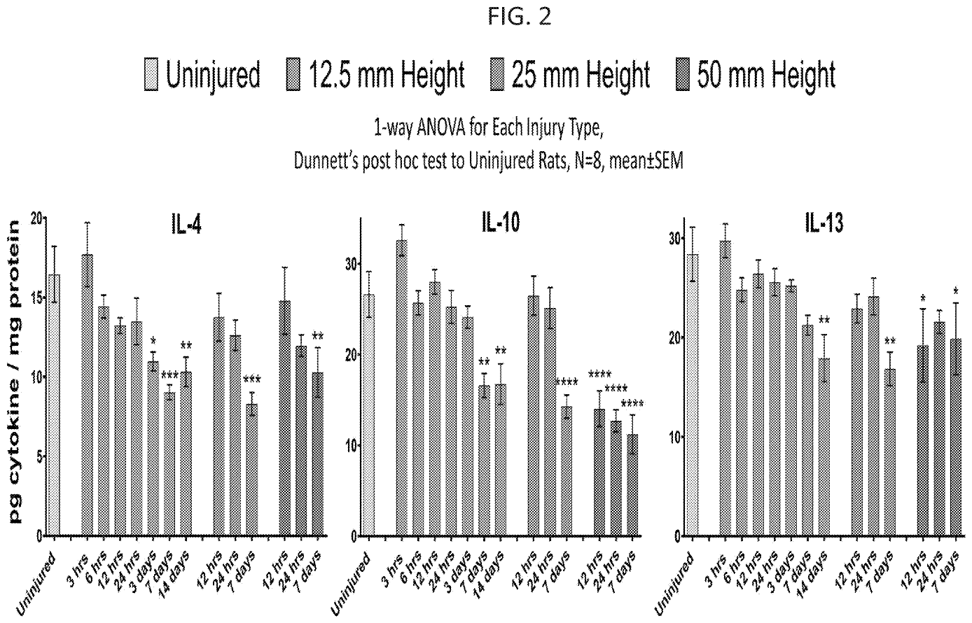

IL-2 is a pleiotropic cytokine that acts as a master regulator and depending on signaling cues from the microenvironment, plays a key role in activation-induced cell death, the formation of cytotoxic T lymphocytes, the secondary expansion of memory CD8⁺ T cells, and the modulation of T helper cell differentiation.⁴⁸ Importantly, IL-2 is vital for development of T_{regs} and IL-2 signaling promotes extensive proliferation and survival of T_{regs}.^{13, 14} However, endogenous IL-2 levels are significantly decreased post-SCI (FIG. 1).¹⁵ T_{regs} constitutively express high IL-2 receptor α-chain but are unable to produce IL-2 and rely on activated T cells for IL-2 production.^{13, 49} The importance of T_{regs} and IL-2 signaling is highlighted by the fact that if mice lack the ability to produce IL-2 or the IL-2 receptor complex, they die from lethal T cell-driven autoimmunity due to reduced functional T_{regs}.⁵⁰⁻⁵³ Increasing IL-2 levels in mice with experimental autoimmune encephalomyelitis (model of multiple sclerosis), reduced neuroinflammation and improved motor function.¹⁴ Moreover, in mice with stroke or traumatic brain injury (TBI), increasing levels of IL-2, reduced inflammation, reduced CNS lesion size, and significantly improved motor function.¹⁴ Moreover, Treg depletion after SCI in mice resulted in significantly worse hind limb motor function.¹⁰

### Therapeutic Strategies for Increasing IL-2 Levels Post-SCI

People living with SCI are prone to pneumonia, skin and soft tissue infections, urinary tract infections, and bloodstream infections. It has also been shown that patients treated with systemic methylprednisolone to reduce inflammation after SCI had a significantly higher rate of complications including urinary tract infections, decubitus ulcers, and pneumonias, as compared to untreated patients.⁵⁴ In addition PMN-MDSCs have a reduced capacity to fight Staphylococcus aureus infections,” and we are hypothesizing TGF-β secretion by T_{regs} will promote immune-suppressive PMN-MDSCs. Therefore, systemic upregulation of IL-2 is not a viable option for treating SCI.

Local delivery of recombinant IL-2 is also not feasible due to its short half-life (~15 minutes) and the need for an intraspinal injection to avoid systemic upregulation.⁵⁶⁻⁵⁸ There are two disadvantages to using an intraspinal injection for IL-2 treatment after SCI: 1) the risk of causing additional damage to the uninjured spinal cord, and 2) the significant time required for preparation and administration of the injection, which is likely to result in missing the early therapeutic window for treatment. To perform an intraspinal injection after human SCI, the patient must be transported to the hospital, stabilized, and undergo imaging before the injection. Thus, the most likely time for the injection would be during spinal stabilization surgery (>6 hours post-injury).^{59, 60} Ideally, IL-2 treatment should begin within the first 2 hours post-SCI, because within this timeframe inflammatory cytokines are being upregulated and neutrophils are infiltrating. This highlights the need for a platform that can locally upregulate IL-2 within the injury site without requiring a specialized intraspinal injection.

Recently, adeno-associated viruses (AAV) combined with a modified GFAP promoter were developed to express IL-2 primarily in astrocytes with limited expression observed in other CNS cells.¹⁴ The IL-2 treatment led to significant improvements in CNS ailments including a model of multiple sclerosis, stroke, and TBI, which demonstrates the efficacy of IL-2 treatment within the CNS.¹⁴ However, gene delivery for SCI treatment using cDNAs or viral vectors such as AAV and lentiviruses, raises safety concerns such as insertional mutagenesis and immunogenic response.⁶¹⁻⁶³ Another shortcoming of the AAV combined with a modified GFAP promoter method when used for SCI treatment is the transfection of astrocytes in unwanted areas of the CNS. Although AAV gene therapy may lead to a higher upregulation of IL-2 near the injury due to localized astrogliosis that occurs after SCI, gene therapy vectors using AAV infect both dividing and quiescent cells, which would upregulate IL-2 to an extent throughout the CNS.⁶⁴

The work described herein addresses drug delivery limitations for the treatment of SCI with IL-2 by utilizing lipid nanoparticles (LNPs) to transfect astrocytes for non-viral upregulation of IL-2 within the injury. mRNA gene delivery has emerged as a strategy for inducing production of therapeutic proteins in situ, offering higher activity than recombinant proteins and a more favorable safety profile compared to viral gene delivery.^{63, 65, 66} Furthermore, the recent rapid development of vaccines for infectious diseases, such as SARS-CoV-2, demonstrates three distinct advantages that are relevant to SCI treatment: 1) LNPs efficiently transfect human cells, 2) mRNAs effectively upregulate a desired protein with minimal immunogenic response, and 3) the clinical relevance of using LNPs to deliver mRNA.^{67, 68} The data collected on the LNPs herein demonstrate the potency of LNPs in delivering mRNA for the treatment of SCI, with effective transfection localized to the injury site.

### Results

Development of mRNA-LNPs with an Astrocyte Specific Biological Circuit (Astro-mRNA-LNPs) for Non-Viral mRNA Transfection

Non-viral mRNA delivery using LNPs is clinically approved and highly effective, as demonstrated by LNP mRNA-based vaccines for infectious diseases such as COVID-19.^{61, 67-69} Non-viral LNP-based mRNA delivery offers several conceptual advantages over protein and other nucleic acid-based approaches, including reduced immune responses, efficient in situ protein production with proper post-translational modifications, simultaneous delivery of multiple genes, and the elimination of the need for nuclear entry.⁷⁰ In addition, LNPs are highly adaptable, and modifications to surface targeting moieties can be made to increase transfection in specific organs or cells of interest.⁷¹⁻⁷⁴ Another emerging strategy in achieving site specific gene delivery is the use of synthetic biological circuits in the LNP nucleic acid payload that respond to cell-type specific signals to ensure control of transgene expression.^{63, 75} Advantageously, the L7Ae system uses miRNA-based logic inputs for controlling gene expression making it highly adaptable for any cell type.⁷⁶ The work herein uses the engineering of LNPs and their payload for efficient astrocyte-specific transfection as a non-viral mRNA delivery method for SCI treatments.

LNP Surface TargetingMoieties: Astrocytes are efficient secretory cells that are prevalent around the SCI site. Based on previous literature, we assembled a library of specific peptides that target astrocytes for incorporation on the surface of LNPs for testing.^{77, 78} We used FDA-approved, commercially available LNP formulations, with the same classification as the COVID-19 mRNA vaccines (FIG. 3).

LNP synthesis: mRNA LNPs were synthesized as follows: An ethanol phase containing an ionizable lipid, structural lipid, cholesterol, and pegylated lipid were prepared at lipid component molar ratios of 50, 10, 38.5 and 1.5 respectively. An aqueous phase containing the payload mRNA was prepared by diluting 1 mg/ml mRNA with 10 mM citrate buffer. The ethanol and aqueous phases were mixed using a microfluidic device. The ratio of lipids to nucleic acid payload was determined by maintaining a set ratio of ionizable nitrogen in the lipids to anionic phosphates in the mRNA backbone of 6:1. For simple surface conjugation of peptides to LNP surfaces, the well-defined maleimide-thiol click chemistry was used, which requires minimal purification steps. A Michael-type addition reaction between a free thiol group and a maleimide group occurs at a pH Range of 6.5-8, resulting in thiosuccinimide bond. The free thiol was incorporated into peptides as a terminal cysteine residue, and the maleimide was incorporated into the LNPs, via a maleimide terminated PEG-lipid.

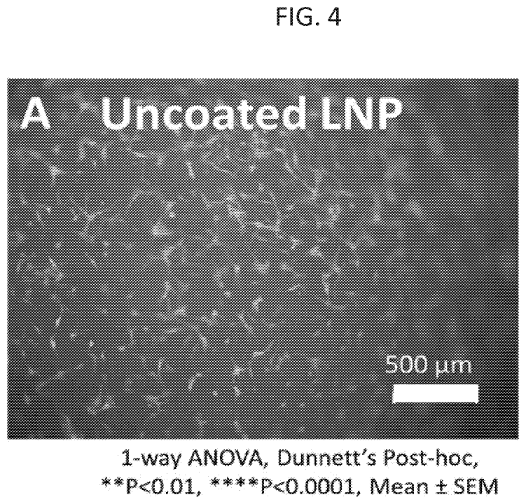

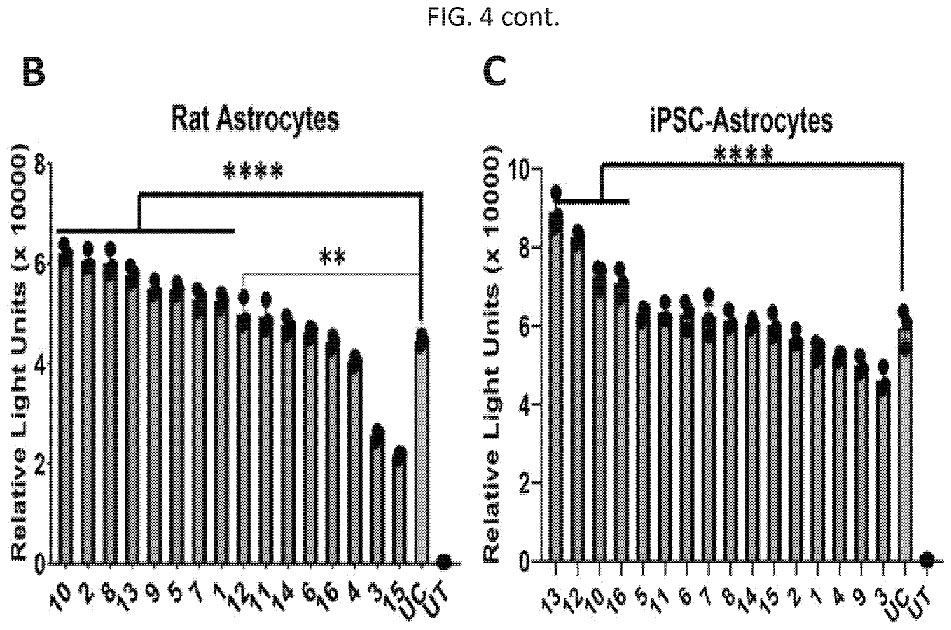

To test astrocyte transfection, LNPs were synthesized, containing enhanced green fluorescent protein (eGFP) mRNA or firefly luciferase (Fluc) mRNA, and incorporating the maleimide-PEG-lipid (0.2 molar ratio) as a fraction of the total PEG-lipid (1.3 molar ratio). Following dialysis, maleimide-containing LNPs were incubated with 10 molar excess of cysteine terminated peptide to maleimide groups. Conjugated LNPs were purified using a second round of dialysis before being added to astrocyte cell cultures in 96 well plates (400 ng mRNA/well). 24 hours after adding the LNPs, the cells were lysed, and luciferin was added before measuring luminescence. Several LNP formulations resulted in significantly higher luminescence compared to Untreated (UT) cells and cells treated with Uncoated (UC) LNPs, demonstrating specific LNP surface peptides that enhance transfection in rat astrocytes and human induced pluripotent stem cell (hiPSC) derived astrocytes (FIG. 4; Table 1)._These results identify specific peptides that target astrocytes and increase transfection.

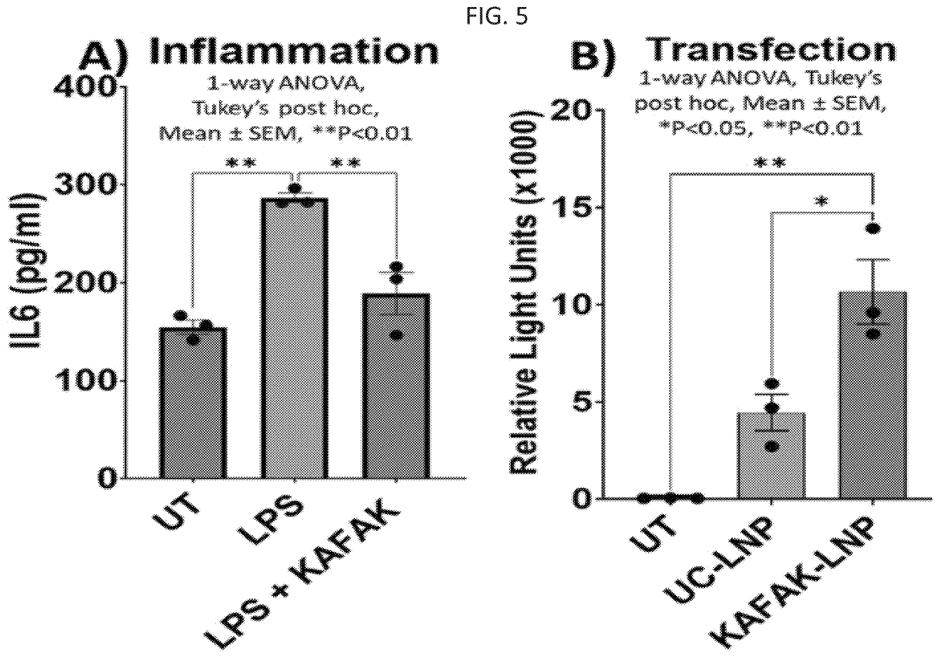

Minimizing Inflammation: Previous work has identified cell-penetrating peptides, one such peptide is derived from the heparin-binding domain of anti-thrombin III, termed KAFAK.⁸⁰ KAFAK not only acts as a cell-penetrating peptide but also confers biological activity as an anti-inflammatory biologic. After cell internalization, KAFAK can target mitogen-activated protein kinase 2 to reduce the expression of pro-inflammatory cytokines, including TNF-α and IL-6.⁸⁰ In addition, KAFAK has been explored as a treatment for SCI, demonstrating potent abilities to reduce inflammation at the site of SCI and improve functional recovery.⁸¹ KAFAK-conjugated mRNA-LNPs have the potential to reduce adjuvant activitys^{82, 83} after LNP endocytosis and provide anti-inflammatory benefits after SCI. To test the anti-inflammatory effects of KAFAK on rat astrocytes, cell cultures were activated with lipopolysaccharide (LPS) and KAFAK was added. KAFAK significantly reduced IL-6 secretion from LPS activated astrocytes (FIG. 5A). To test transfection of KAFAK-conjugated LNPs, the KAFAK peptide was conjugated as described above in section “LNP Surface Targeting Moieties”. KAFAK-conjugated LNPs loaded with Fluc mRNA significantly increased astrocyte transfection compared to standard UC LNPs (FIG. 5B). These results demonstrate the anti-inflammatory capabilities of KAFAK and highlight KAFAK-conjugated LNPs potential for use after SCI.

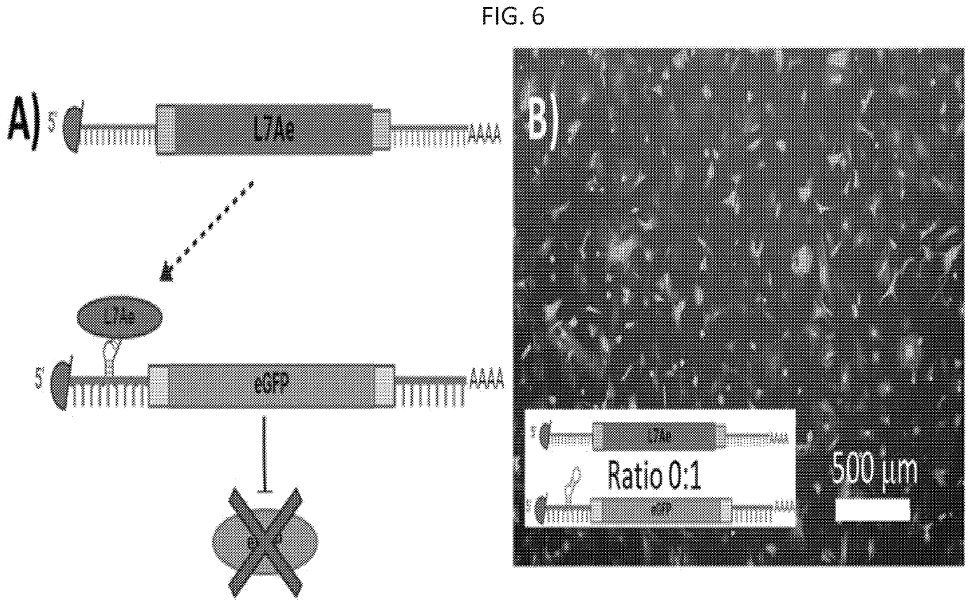

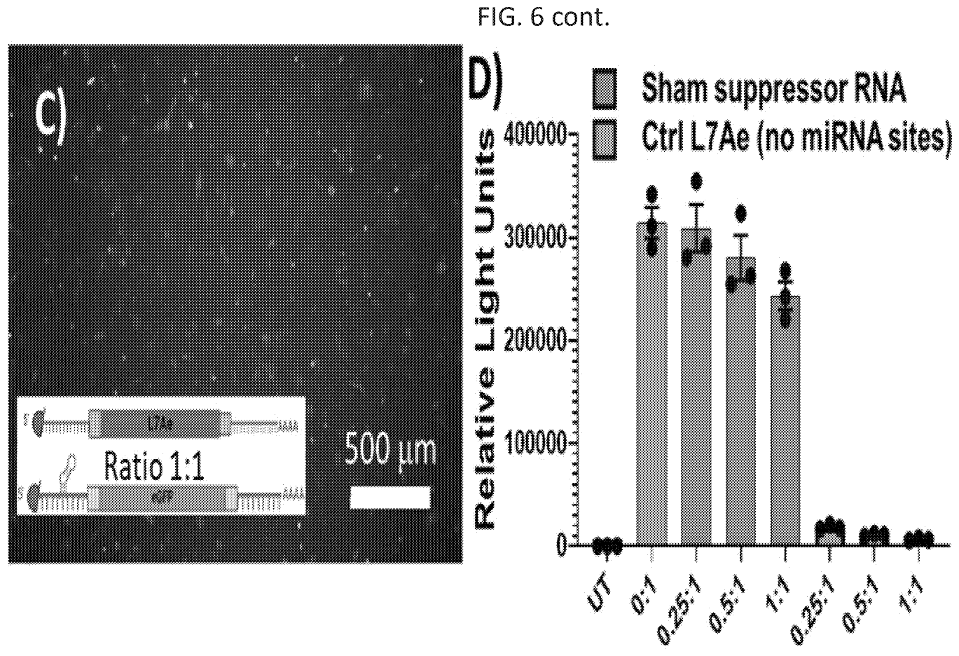

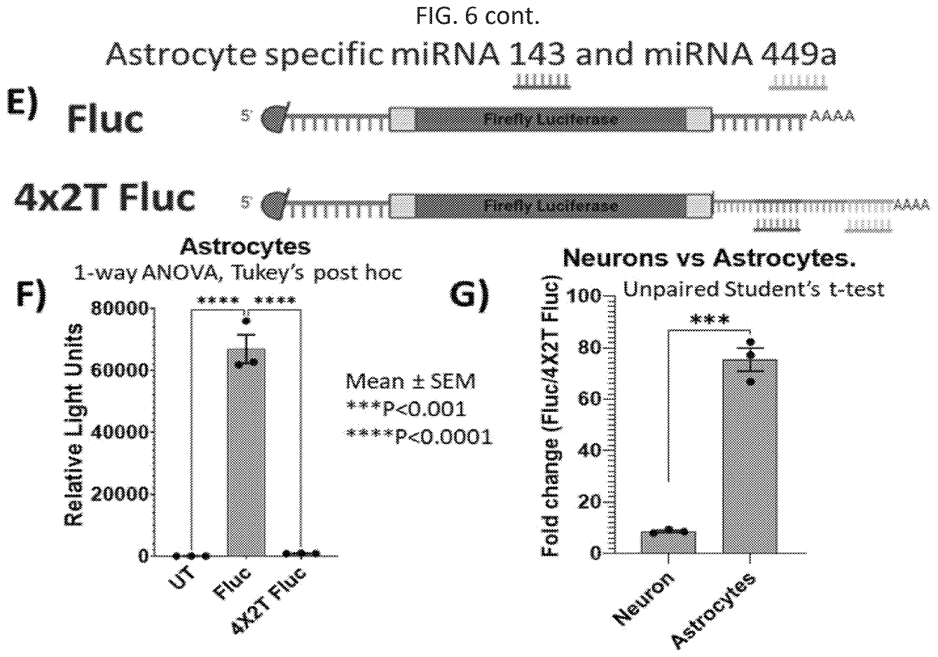

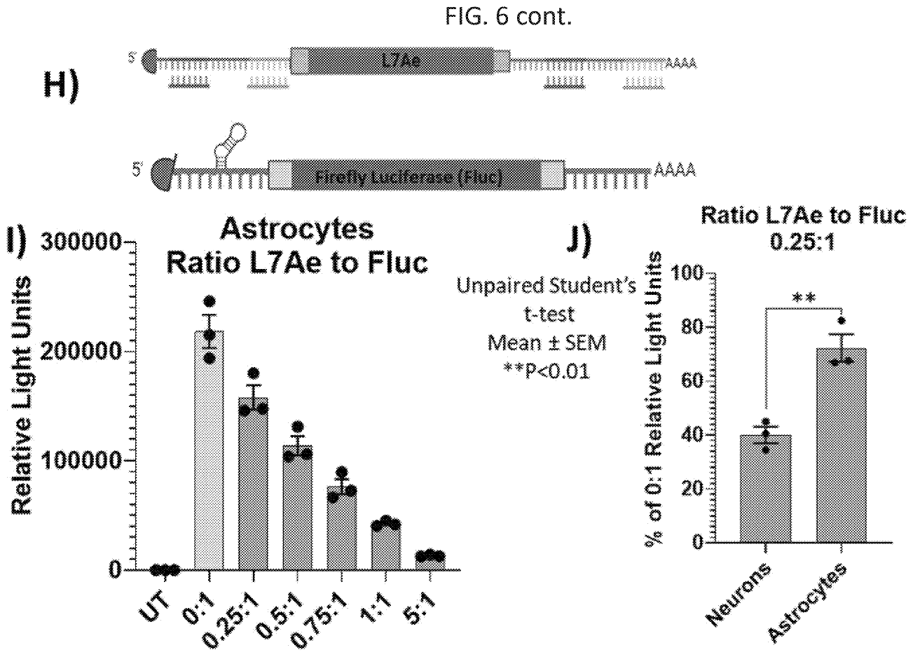

miRNA-Based Logic for Controlling Gene Expression: The miRNA-based logic circuit L7Ae system for controlling gene expression utilizes a secondary mRNA, termed the ‘suppressor’ strand, which is co-delivered with the therapeutic mRNA strand containing the transgene of interest.⁶³ The suppressor mRNA encodes an archaeal protein (L7Ae) that binds with high specificity to the secondary structure motif of a unique RNA sequence termed a “K-turn.” This K-turn sequence is inserted into the 5′ UTR of the therapeutic mRNA strand, where binding of L7Ae blocks ribosomal translation of the transgene (FIG. 6A-D). Cell-type-specific expression of the transgene is achieved by including cell-type-specific miRNA target sites in the 3′ UTR and/or 5′ UTR of the suppressor mRNA strand. Thus, when the dual mRNA strands are delivered to the target cell containing the specified miRNA, the suppressor strand degrades, allowing the therapeutic mRNA to express the transgene of interest. However, when the dual strands are delivered to off-target cells lacking the specific miRNA, the suppressor strand does not degrade, and L7Ae suppresses the delivered transgene.

We use the L7Ae system with astrocyte specific mir-449a and mir-143⁸⁴ by incorporating their exact complementary sequences into the 3′UTR and 5′UTR region of the L7Ae suppressor mRNA (FIG. 6E-G). We incorporated a K-turn motif for L7Ae protein binding into the 5′UTR of multiple reporter genes. For in vitro validation, we transfected rat primary neurons and astrocytes with L7Ae Fluc-mRNA LNPs co-delivering the K-turn-reporter mRNA and the L7Ae-miRNA-suppressor.

Fluc mRNA with 4 repeats of mir-449a and mir-143 sites in its 3′UTR transfected neurons but not astrocytes. This demonstrates the miRNA was indeed in astrocytes and the Fluc mRNA was degraded, and that the miRNA was not in neurons allowing for Fluc translation (FIG. 6E-G). Even at a low ratio of L7Ae to Fluc mRNA, the L7Ae repressor mRNA, lacking mir-449a and mir-143 sites, almost completely inhibited the translation of the Fluc mRNA in astrocytes (FIG. 6D). However, when the L7Ae repressor mRNA contained four repeats of mir-449a and mir-143 sites in both 3′ UTR and 5′ UTR, there was significantly more transfection in astrocytes than neurons (FIG. 6H-J). Even at a low ratio of L7Ae (0.25 to 1), neuron transfection was suppressed by ~60% (FIG. 6J). These results demonstrate the capability of the L7Ae system with astrocyte specific miRNA-based logic (FIG. 6H) to promote astrocyte transfection while restricting transfection in neurons. We will test and develop this specific L7Ae miRNA circuit for IV administration following SCI.

### Localized In Vivo Transfection Post-SCI to Reduce Neuroinflammation

Previous studies have shown that polymeric nanoparticles of a similar size to LNPs can accumulate at the lesion site of SCI following IV administration.⁸⁵ It has also been demonstrated that mRNA-LNPs can transfect the spinal cord following local injections,⁸⁶ though their ability to transfect the SCI following IV administration has yet to be explored. During the acute phase, broken vasculature exposes the spinal cord parenchyma to circulating blood, triggering a localized immune response.^{15, 87} In the sub-acute and chronic stages, an astrocytic border forms around the lesion site as the body's mechanism to isolate the healthy parenchyma from systemic circulation.^{3, 27, 28} Given that the vasculature at the lesion site remains compromised for some time after injury,⁸⁷ different timepoints could be explored for IV administration of mRNA-LNPs to assess the potential of gene therapies for treating various pathological stages of SCI.

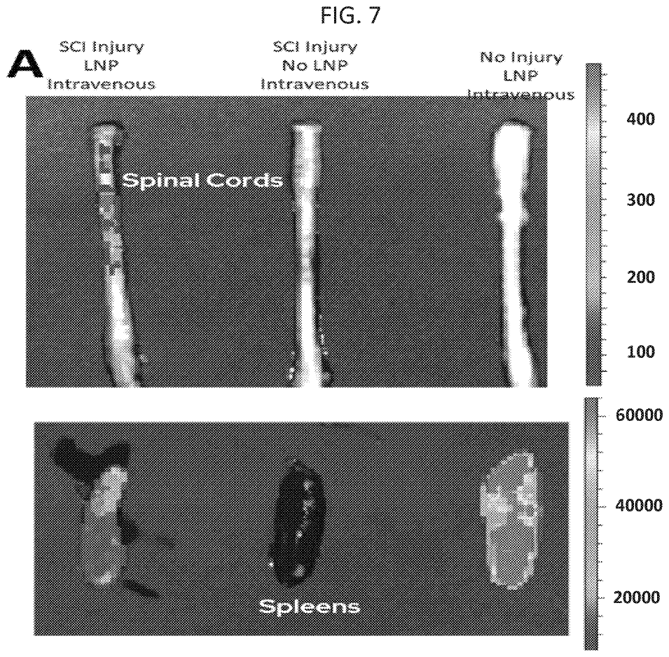

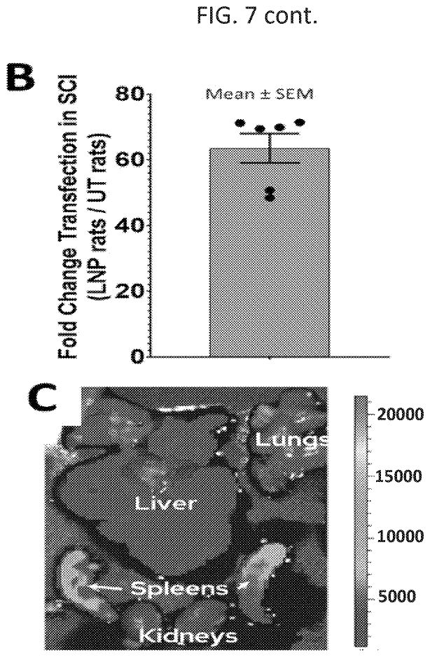

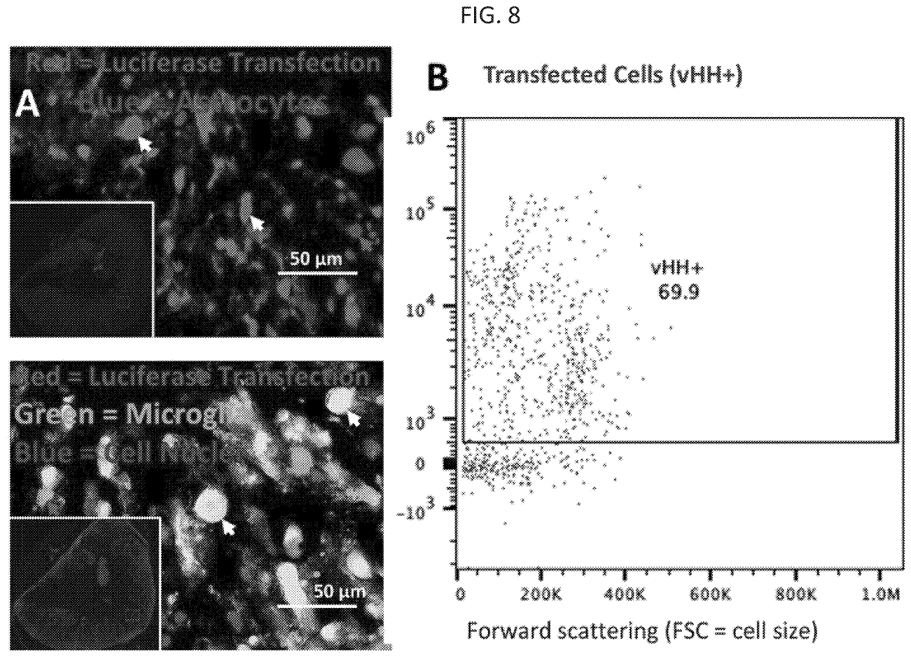

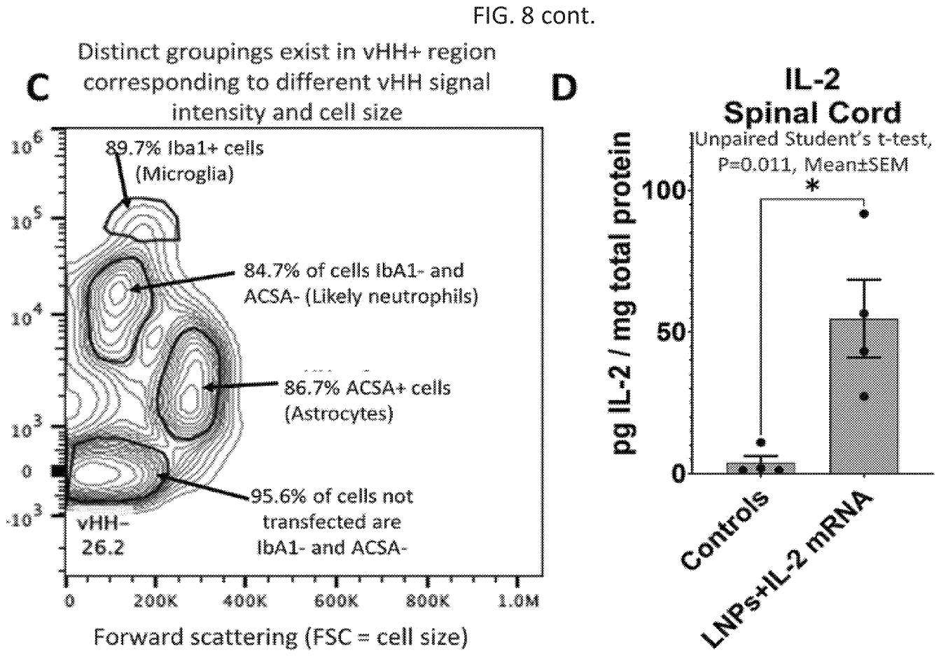

We performed a series of experiments that demonstrate a high potential for success at developing a new approach for transfecting cells directly within the injured spinal cord after IV administration. The following capabilities are essential for the success and broad applicability of this approach: 1) LNPs are able to cross the disrupted BSCB but not the intact BSCB, 2) LNPs effectively transfect CNS-specific cells, and 3) LNPs are capable of upregulating IL-2 directly in the injured spinal cord. We have conducted experiments in Sprague Dawley (SD) rats with jugular vein catheters (JVC) that demonstrate success in each of these critical capabilities. For these experiments SD rats with JVCs were purchased, subjected to a T10 contusion SCI, LNPs were administered IV 30 minutes post-injury, and transfection was assessed 24 hours post-injury. First, following IV administration, LNPs directly transfected the spinal cord after injury. After IV administration of Fluc mRNA-loaded LNPs there was over a 60-fold increase in luminescence compared to SCI rats without LNP injection (FIG. 7A-B). Importantly, in the injured rats, trans-fection in the spinal cord was localized to the injured tissue, and no transfection was observed in the CNS of uninjured rats after the same LNP treatment (FIG. 7A-C). These results confirm that LNPs can enter the spinal cord through damaged vasculature after injury. Second, immune-histochemistry (IHC) on transverse sections revealed excellent transfection of astrocytes and microglia after IV administration of Fluc mRNA-loaded LNPs post-SCI (FIG. 8A). Additionally, we assessed transfected cells with flow cytometry using a membrane-bound reporter gene encoding for a camelid variable heavy chain (vHH) nanobody that is anchored via a glycosylphosphatidylinositol lipid. Flow cytometry performed on single cell suspensions of ~7 mm of spinal cord centered on the injury showed that almost 70% of the cells were transfected including over 90% of all astrocytes were transfected (FIG. 8B-C). Third, IL-2 mRNA loaded LNPs significantly increased IL-2 levels in the spinal cord (FIG. 8D). Importantly, these results demonstrate that LNPs given IV cross the disrupted BSCB and transfect astrocytes in the SCI to upregulate IL-2. The development of the L7Ae miRNA circuit may minimize off target transfection shown in FIG. 7C. Previous literature showing IL-2's ability to upregulate T_{regs} and the immunosuppressive capability of T_{regs}, along with our combined work here demonstrating non-viral transfection with LNPs and upregulation of IL-2 after SCI, establishes a high propensity for success in this work.

The characteristics of inflammation following SCI, along with the role of immunosuppressive T_{regs} and IL-2 signaling to promote T_{reg} proliferation, strongly suggest that local transfection and upregulation of IL-2 can enhance T_{reg} populations, thereby reducing inflammation after SCI. Our work demonstrates that we can enhance astrocyte transfection by incorporating specific peptides onto LNPs, reduce inflammation by adding the KAFAK peptide to LNPs, and directly upregulate IL-2 in the injured spinal cord. However, our work was conducted as proof of concept. While it clearly demonstrates the capabilities of Astro-mRNA-LNPs, studies are still needed to maximize their efficiency and test their ability to improve function after SCI. Additionally, the standard Uncoated LNPs tested in vivo performed excellent in transfecting the injured spinal cord, but it also transfected the liver and spleen (FIG. 7C). This highlights the need to implement the L7Ae mRNA repressor circuit to minimize off-target transfection. We have strong in vitro data showing the ability of the L7Ae circuit to reduce transfection in unwanted cells while still effectively transfecting astrocytes (FIG. 6). Our results provide important evidence that Astro-mRNA-LNPs can enhance transfection in the injured spinal cord after IV administration. The guiding premise of the research discussed herein is that current drug delivery limitations for the treatment of SCI with IL-2 can be addressed by utilizing Astro-mRNA-LNPs to non-virally upregulate IL-2 directly in the injury.

### Materials and Methods

### Lipid Nanoparticle Formulation

Lipid nanoparticle formulation described in the Moderna SPIKEVAX COVID-19 mRNA vaccine is used. Briefly, the LNP consists of the following lipid components formulated at the indicated molar ratios:
Ionizable lipid: SM-102-50% molar ratio
Structural lipid: 1,2-Distearoyl-sn-glycero-3-PC (1,2-DSPC)—10% Molar ratio
Sterol: Cholesterol—38.5% molar ratio
PEGylated lipid: 1,2-dimyristoyl-rac-glycero-3-methoxypolyethylene glycol-2000 (DMG-PEG2000)—1.5% molar ratio

When peptides are conjugated to the LNP, a 5th lipid component is included in the lipid phase:
Maleimide-PEGylated lipid: 1,2-distearoyl-sn-glycero-3-phosphoethanolamine-N-[maleimide (polyethylene glycol)-2000](ammonium salt)

The molar ratios of the PEGylated lipid and Maleimide-PEGylated lipid are still being optimized, though to date we have been using 1.3% and 0.2% ratios respectively, to add up to a 1.5% total PEG-lipid molar ratio in the LNP.

### Protocol

Lipids components are combined in an ethanol phase, and nucleic acids are diluted in a 10Mm Citrate buffer at pH 4. The ethanol phase is mixed with the aqueous phase at a volume feed ratio of 1:3 and a nitrogen (ionizable lipid) to phosphate (mRNA backbone) ratio of 6. LNPs are then dialysized against PBS prior to in vitro or in vivo administration.

### Peptide Conjugation

Following dialysis of mRNA-LNPs formulated with Maleimide-PEG-lipids, the LNPs dispersed in PBS are transferred to a collection tube and incubated with 10× molar excess (in relation to Maleimide PEG lipids) of cysteine-terminated peptides for 2 hr. Following peptide incubation, conjugated LNPs are put on dialysis for a second time overnight at 4° C. Final LNPs are transferred to collections tubes prior to administration.

TABLE 1
Peptides tested

| | | | MW | Rat | IPSC |
|---|---|---|---|---|---|
| Number | Peptide | Sequence | (g/mol) | Astrocytes | astrocytes |
| 1 | AS1-N | CLNSSQPS (SEQ ID NO: 1) | 834.9 | P < 0.0001 | ns |
| 2 | AS1-C | LNSSQPSC (SEQ ID NO: 2) | 834.9 | P < 0.0001 | ns |
| 3 | A-C3-5-N | CSAYLDSLN (SEQ ID NO: 3) | 985.07 | ns | ns |
| 4 | A-C3-5-C | SAYLDSLNC (SEQ ID NO: 4) | 985.07 | ns | ns |
| 5 | A-21-1-N | CSGNSTSNAC (SEQ ID NO: 5) | 839.83 | P < 0.0001 | ns |
| 6 | A-21-1-C | SGNSTSNAC (SEQ ID NO: 6) | 839.83 | ns | ns |
| 7 | A+C1-11-N | CSGSMIAMP (SEQ ID NO: 7) | 896.11 | P < 0.0001 | ns |
| 8 | A+C1-11-C | SGSMIAMPC (SEQ ID NO: 8) | 896.11 | P < 0.0001 | ns |
| 9 | A+C4-9-N | CSPLSFQAT (SEQ ID NO: 9) | 953.08 | P < 0.0001 | ns |
| 10 | A+C4-9-C | SPLSFQATC (SEQ ID NO: 10) | 953.08 | P < 0.0001 | P < 0.0001 |
| 11 | ADC6-5-N | CSGSSFTMI (SEQ ID NO: 11) | 932.08 | P = 0.0267 | ns |
| 12 | ADC6-5-C | SGSSFTMIC (SEQ ID NO: 12) | 932.08 | P = 0.0100 | P < 0.0001 |
| 13 | AC2-6-N | CSPNTNTLA (SEQ ID NO: 13) | 920 | P < 0.0001 | P < 0.0001 |
| 14 | AC2-6-C | SPNTNTLAC (SEQ ID NO: 14) | 920 | ns | ns |
| 15 | A+C5-1-N | CSVDDYFTR (SEQ ID NO: 15) | 1105.18 | ns | ns |
| 16 | A+C5-1-C | SVDDYFTRC (SEQ ID NO: 16) | 1105.18 | ns | P = 0.008 |
| 17 | KAFAK | KAFAKLAARLYRKALARQLG | | Not tested | P = 0.0128 |
| | | VAA (SEQ ID NO: 17) | | | |

### REFERENCES

1. (NSCIS) NSCISC. Spinal cord injury (SCI) facts and figures at a glance: 2020 SCI Data SheetLast access August 2022. doi: https//www.nscisc.uab.edu/Public/Facts FIG. 2020.pdf.
2. Kwiecien J M, Dabrowski W, Dabrowska-Bouta B, Sulkowski G, Oakden W, Kwiecien-Delaney C J, Yaron J R, Zhang L, Schutz L, Marzec-Kotarska B, Stanisz G J, Karis J P, Struzynska L, Lucas A R. Prolonged inflammation leads to ongoing damage after spinal cord injury. PLoS One. 2020; 15(3):e0226584. Epub 20200319. doi: 10.1371/joumnal.pone.0226584. PubMed PMID: 32191733; PMCID: PMC7081990.
3. Hellenbrand D J, Quinn C M, Piper Z J, Morehouse C N, Fixel J A, Hanna A S. Inflammation after spinal cord injury: a review of the critical timeline of signaling cues and cellular infiltration. J Neuroinflammation. 2021; 18(1):284. Epub 20211207. doi: 10.1186/s12974-021-02337-2. PubMed PMID: 34876174; PMCID: PMC8653609.
4. Zhang Q, Yu B, Zhang Y, Tian Y, Yang S, Chen Y, Wu H. Combination of single-cell and bulk RNA seq reveals the immune infiltration landscape and targeted therapeutic drugs in spinal cord injury. Front Immunol. 2023; 14:1068359. Epub 20230119. doi: 10.3389/fimmu.2023.1068359. PubMed PMID: 36742334; PMCID: PMC9894719.
5. Liu C, Chikina M, Deshpande R, Menk A V, Wang T, Tabib T, Brunazzi E A, Vignali K M, Sun M, Stolz D B, Lafyatis R A, Chen W, Delgoffe G M, Workman C J, Wendell S G, Vignali D A A. Treg Cells Promote the SREBP1-Dependent Metabolic Fitness of Tumor-Promoting Macrophages via Repression of CD8(+) T Cell-Derived Interferon-gamma. Immunity. 2019; 51(2):381-97 e6. Epub 20190723. doi: 10.1016/j.immuni.2019.06.017. PubMed PMID: 31350177; PMCID: PMC6703933.
6. Zhao H, Liao X, Kang Y. Tregs: Where We Are and What Comes Next? Front Immunol. 2017; 8:1578. Epub 20171124. doi: 10.3389/fimmu.2017.01578. PubMed PMID: 29225597; PMCID: PMC5705554.
7. Fridlender Z G, Sun J, Kim S, Kapoor V, Cheng G, Ling L, Worthen G S, Albelda S M. Polarization of tumor-associated neutrophil phenotype by TGF-beta: “N1” versus “N2” TAN. Cancer Cell. 2009; 16(3):183-94. doi: 10.1016/j.ccr.2009.06.017. PubMed PMID: 19732719; PMCID: PMC2754404.
8. Guan T, Zhou X, Zhou W, Lin H. Regulatory T cell and macrophage crosstalk in acute lung injury: future perspectives. Cell Death Discov. 2023; 9(1):9. Epub 20230116. doi: 10.1038/s41420-023-01310-7. PubMed PMID: 36646692; PMCID: PMC9841501.
9. Sagiv J Y, Michaeli J, Assi S, Mishalian I, Kisos H, Levy L, Damti P, Lumbroso D, Polyansky L, Sionov R V, Ariel A, Hovav A H, Henke E, Fridlender Z G, Granot Z. Phenotypic diversity and plasticity in circulating neutrophil subpopulations in cancer. Cell Rep. 2015; 10(4):562-73. Epub 20150122. doi: 10.1016/j.celrep.2014.12.039. PubMed PMID: 25620698.
10. Liu R, Li Y, Wang Z, Chen P, Xie Y, Qu W, Wang M, Yu Z, Luo X. Regulatory T cells promote functional recovery after spinal cord injury by alleviating microglia inflammation via STAT3 inhibition. CNS Neurosci Ther. 2023; 29(8):2129-44. Epub 20230313. doi: 10.1111/cns.14161. PubMed PMID: 36914969; PMCID: PMC10352886.
11. Dombrowski Y, O'Hagan T, Dittmer M, Penalva R, Mayoral S R, Bankhead P, Fleville S, Eleftheriadis G, Zhao C, Naughton M, Hassan R, Moffat J, Falconer J, Boyd A, Hamilton P, Allen I V, Kissenpfennig A, Moynagh P N, Evergren E, Perbal B, Williams A C, Ingram R J, Chan J R, Franklin R J M, Fitzgerald D C. Regulatory T cells promote myelin regeneration in the central nervous system. Nat Neurosci. 2017; 20(5):674-80. Epub 20170313. doi: 10.1038/nn.4528. PubMed PMID: 28288125; PMCID: PMC5409501.
12. de la Fuente A G, Dittmer M, Heesbeen E J, de la Vega Gallardo N, White J A, Young A, McColgan T, Dashwood A, Mayne K, Cabeza-Fernandez S, Falconer J, Rodriguez-Baena F J, McMurran C E, Inayatullah M, Rawji K S, Franklin R I M, Dooley J, Liston A, Ingram R J, Tiwari V K, Penalva R, Dombrowski Y, Fitzgerald D C. Ageing impairs the regenerative capacity of regulatory T cells in mouse central nervous system remyelination. Nat Commun. 2024; 15(1):1870. Epub 20240311. doi: 10.1038/s41467-024-45742-w. PubMed PMID: 38467607; PMCID: PMC10928230.
13. Chinen T, Kannan A K, Levine A G, Fan X, Klein U, Zheng Y, Gasteiger G, Feng Y, Fontenot J D, Rudensky A Y. An essential role for the IL-2 receptor in T(reg) cell function. Nat Immunol. 2016; 17(11):1322-33. Epub 20160905. doi: 10.1038/ni.3540. PubMed PMID: 27595233; PMCID: PMC5071159.
14. Yshii L, Pasciuto E, Bielefeld P, Mascali L, Lemaitre P, Marino M, Dooley J, Kouser L, Verschoren S, Lagou V, Kemps H, Gervois P, de Boer A, Burton O T, Wahis J, Verhaert J, Tareen S H K, Roca C P, Singh K, Whyte C E, Kerstens A, Callaerts-Vegh Z, Poovathingal S, Prezzemolo T, Wierda K, Dashwood A, Xie J, Van Wonterghem E, Creemers E, Aloulou M, Gsell W, Abiega O, Munck S, Vandenbroucke R E, Bronckaers A, Lemmens R, De Strooper B, Van Den Bosch L, Himmelreich U, Fitzsimons C P, Holt M G, Liston A. Astrocyte-targeted gene delivery of interleukin 2 specifically increases brain-resident regulatory T cell numbers and protects against pathological neuroinflammation. Nat Immunol. 2022; 23(6):878-91. Epub 20220526. doi: 10.1038/s41590-022-01208-z. PubMed PMID: 35618831; PMCID: PMC9174055.
15. Hellenbrand D J, Quinn C M, Piper Z J, Elder R T, Mishra R R, Marti T L, Omuro P M, Roddick R M, Lee J S, Murphy W L, Hanna A S. The secondary injury cascade after spinal cord injury: an analysis of local cytokine/chemokine regulation. Neural Regen Res. 2024; 19(6):1308-17. Epub 20230922. doi: 10.4103/1673-5374.385849. PubMed PMID: 37905880; PMCID: PMC11467934.
16. Mortazavi M M, Verma K, Harmon O A, Griessenauer C J, Adeeb N, Theodore N, Tubbs R S. The microanatomy of spinal cord injury: a review. Clin Anat. 2015; 28(1):27-36. Epub 20140711. doi: 10.1002/ca.22432. PubMed PMID: 25044123.
17. Gensel J C, Kopper T J, Zhang B, Orr M B, Bailey W M. Predictive screening of M1 and M2 macrophages reveals the immunomodulatory effectiveness of post spinal cord injury azithromycin treatment. Sci Rep. 2017; 7:40144. Epub 20170106. doi: 10.1038/srep40144. PubMed PMID: 28057928; PMCID: PMC5216345.
18. Brennan F H, Li Y, Wang C, Ma A, Guo Q, Li Y, Pukos N, Campbell W A, Witcher K G, Guan Z, Kigerl K A, Hall J C E, Godbout J P, Fischer A J, McTigue D M, He Z, Ma Q, Popovich P G. Microglia coordinate cellular interactions during spinal cord repair in mice. Nat Commun. 2022; 13(1):4096. Epub 20220714. doi: 10.1038/s41467-022-31797-0. PubMed PMID: 35835751; PMCID: PMC9283484.
19. Taoka Y, Okajima K, Uchiba M, Murakami K, Kushimoto S, Johno M, Naruo M, Okabe H, Takatsuki K. Role of neutrophils in spinal cord injury in the rat. Neuroscience. 1997; 79(4):1177-82. doi: 10.1016/s0306-4522(97)00011-0. PubMed PMID: 9219976.
20. Stirling D P, Liu S, Kubes P, Yong V W. Depletion of Ly6G/Gr-1 leukocytes after spinal cord injury in mice alters wound healing and worsens neurological outcome. J Neurosci. 2009; 29(3):753-64. doi: 10.1523/JNEUROSCI.4918-08.2009. PubMed PMID: 19158301; PMCID: PMC6665178.
21. Garcia E, Aguilar-Cevallos J, Silva-Garcia R, Ibarra A. Cytokine and Growth Factor Activation In Vivo and In Vitro after Spinal Cord Injury. Mediators Inflamm. 2016; 2016:9476020. Epub 20160623. doi: 10.1155/2016/9476020. PubMed PMID: 27418745; PMCID: PMC4935915.
22. Popovich P G, van Rooijen N, Hickey W F, Preidis G, McGaughy V. Hematogenous macrophages express CD8 and distribute to regions of lesion cavitation after spinal cord injury. Exp Neurol. 2003; 182(2):275-87. doi: 10.1016/s0014-4886(03)00120-1. PubMed PMID: 12895439.
23. Kim S U, de Vellis J. Microglia in health and disease. J Neurosci Res. 2005; 81(3):302-13. doi: 10.1002/jnr.20562. PubMed PMID: 15954124.
24. Beck K D, Nguyen H X, Galvan M D, Salazar D L, Woodruff T M, Anderson A J. Quantitative analysis of cellular inflammation after traumatic spinal cord injury: evidence for a multiphasic inflammatory response in the acute to chronic environment. Brain. 2010; 133(Pt 2):433-47. doi: 10.1093/brain/awp322. PubMed PMID: 20085927; PMCID: 2858013.
25. Bellver-Landete V, Bretheau F, Mailhot B, Vallieres N, Lessard M, Janelle M E, Vernoux N, Tremblay M E, Fuehrmann T, Shoichet M S, Lacroix S. Microglia are an essential component of the neuroprotective scar that forms after spinal cord injury. Nat Commun. 2019; 10(1):518. Epub 20190131. doi: 10.1038/s41467-019-08446-0. PubMed PMID: 30705270; PMCID: PMC6355913.
26. Okada S, Hara M, Kobayakawa K, Matsumoto Y, Nakashima Y. Astrocyte reactivity and astrogliosis after spinal cord injury. Neurosci Res. 2018; 126:39-43. doi: 10.1016/j.neures.2017.10.004. PubMed PMID: 29054466.
27. Faulkner J R, Herrmann J E, Woo M J, Tansey K E, Doan N B, Sofroniew M V. Reactive astrocytes protect tissue and preserve function after spinal cord injury. J Neurosci. 2004; 24(9):2143-55. doi: 10.1523/JNEUROSCI.3547-03.2004. PubMed PMID: 14999065; PMCID: PMC6730429.
28. Sofroniew M V. Astrocyte barriers to neurotoxic inflammation. Nat Rev Neurosci. 2015; 16(5):249-63. doi: 10.1038/nrn3898. PubMed PMID: 25891508; PMCID: PMC5253239.
29. Hao A J, Dheen S T, Ling E A. Expression of macrophage colony-stimulating factor and its receptor in microglia activation is linked to teratogen-induced neuronal damage. Neuroscience. 2002; 112(4):889-900. doi: 10.1016/s0306-4522(02)00144-6. PubMed PMID: 12088748.
30. Gratchev A, Schledzewski K, Guillot P, Goerdt S. Alternatively activated antigen-presenting cells: molecular repertoire, immune regulation, and healing. Skin Pharmacol Appl Skin Physiol. 2001; 14(5):272-9. doi: 10.1159/000056357. PubMed PMID: 11586068.
31. Lazarov-Spiegler O, Solomon A S, Zeev-Brann A B, Hirschberg D L, Lavie V, Schwartz M. Transplantation of activated macrophages overcomes central nervous system regrowth failure. FASEB J. 1996; 10(11):1296-302. doi: 10.1096/fasebj.10.11.8836043. PubMed PMID: 8836043.
32. Rapalino O, Lazarov-Spiegler O, Agranov E, Velan G J, Yoles E, Fraidakis M, Solomon A, Gepstein R, Katz A, Belkin M, Hadani M, Schwartz M. Implantation of stimulated homologous macrophages results in partial recovery of paraplegic rats. Nat Med. 1998; 4(7):814-21. doi: 10.1038/nm0798-814. PubMed PMID: 9662373.
33. Rutschman R, Lang R, Hesse M, Ihle I N, Wynn T A, Murray P J. Cutting edge: Stat6-dependent substrate depletion regulates nitric oxide production. J Immunol. 2001; 166(4):2173-7. doi: 10.4049/jimmunol.166.4.2173. PubMed PMID: 11160269.
34. Gensel J C, Zhang B. Macrophage activation and its role in repair and pathology after spinal cord injury. Brain Res. 2015; 1619:1-11. Epub 20150108. doi: 10.1016/j.brainres.2014.12.045. PubMed PMID: 25578260.
35. Bettelli E, Carrier Y, Gao W, Korn T, Strom T B, Oukka M, Weiner H L, Kuchroo V K. Reciprocal developmental pathways for the generation of pathogenic effector TH17 and regulatory T cells. Nature. 2006; 441(7090):235-8. Epub 20060430. doi: 10.1038/nature04753. PubMed PMID: 16648838.
36. Liston A, Pasciuto E, Fitzgerald D C, Yshii L. Brain regulatory T cells. Nat Rev Immunol. 2024; 24(5):326-37. Epub 20231201. doi: 10.1038/s41577-023-00960-z. PubMed PMID: 38040953.
37. Pasciuto E, Burton O T, Roca C P, Lagou V, Rajan W D, Theys T, Mancuso R, Tito R Y, Kouser L, Callaerts-Vegh Z, de la Fuente A G, Prezzemolo T, Mascali L G, Brajic A, Whyte C E, Yshii L, Martinez-Muriana A, Naughton M, Young A, Moudra A, Lemaitre P, Poovathingal S, Raes J, De Strooper B, Fitzgerald D C, Dooley J, Liston A. Microglia Require CD4 T Cells to Complete the Fetal-to-Adult Transition. Cell. 2020; 182(3):625-40 e24. Epub 20200722. doi: 10.1016/j.cell.2020.06.026. PubMed PMID: 32702313; PMCID: PMC7427333.
38. Lewkowicz P, Lewkowicz N, Sasiak A, Tchorzewski H. Lipopolysaccharide-activated CD4+CD25+ T regulatory cells inhibit neutrophil function and promote their apoptosis and death. J Immunol. 2006; 177(10):7155-63. doi: 10.4049/jimmunol.177.10.7155. PubMed PMID: 17082633.
39. Pettinella F, Mariotti B, Lattanzi C, Bruderek K, Donini M, Costa S, Marini O, Iannoto G, Gasperini S, Caveggion E, Castellucci M, Calzetti F, Bianchetto-Aguilera F, Gardiman E, Giani M, Dusi S, Cantini M, Vassanelli A, Pavone D, Milella M, Pilotto S, Biondani P, Hoing B, Schleupner M C, Hussain T, Hadaschik B, Kaspar C, Visco C, Tecchio C, Koenderman L, Bazzoni F, Tamassia N, Brandau S, Cassatella M A, Scapini P. Surface CD52, CD84, and PTGER2 mark mature PMN-MDSCs from cancer patients and G-CSF-treated donors. Cell Rep Med. 2024; 5(2):101380. Epub 20240118. doi: 10.1016/j.xcrm.2023.101380. PubMed PMID: 38242120; PMCID: PMC10897522.
40. El Kebir D, Filep J G. Modulation of Neutrophil Apoptosis and the Resolution of Inflammation through beta2 Integrins. Front Immunol. 2013; 4:60. Epub 20130306. doi: 10.3389/fimmu.2013.00060. PubMed PMID: 23508943; PMCID: PMC3589696.
41. D'Alessio F R, Tsushima K, Aggarwal N R, West E E, Willett M H, Britos M F, Pipeling M R, Brower R G, Tuder R M, McDyer J F, King L S. CD4+CD25+Foxp3+ Tregs resolve experimental lung injury in mice and are present in humans with acute lung injury. J Clin Invest. 2009; 119(10):2898-913. Epub 20090921. doi: 10.1172/JCI36498. PubMed PMID: 19770521; PMCID: PMC2752062.
42. Nakamura K, Kitani A, Strober W. Cell contact-dependent immunosuppression by CD4(+)CD25(+) regulatory T cells is mediated by cell surface-bound transforming growth factor beta. J Exp Med. 2001; 194(5):629-44. doi: 10.1084/jem.194.5.629. PubMed PMID: 11535631; PMCID: PMC2195935.
43. Montaldo E, Lusito E, Bianchessi V, Caronni N, Scala S, Basso-Ricci L, Cantaffa C, Masserdotti A, Barilaro M, Barresi S, Genua M, Vittoria F M, Barbiera G, Lazarevic D, Messina C, Xue E, Marktel S, Tresoldi C, Milani R, Ronchi P, Gattillo S, Santoleri L, Di Micco R, Ditadi A, Belfiori G, Aleotti F, Naldini M M, Gentner B, Gardiman E, Tamassia N, Cassatella M A, Hidalgo A, Kwok I, Ng L G, Crippa S, Falconi M, Pettinella F, Scapini P, Naldini L, Ciceri F, Aiuti A, Ostuni R. Cellular and transcriptional dynamics of human neutrophils at steady state and upon stress. Nat Immunol. 2022; 23(10):1470-83. Epub 20220922. doi: 10.1038/s41590-022-01311-1. PubMed PMID: 36138183; PMCID: PMC7615267.
44. Silvestre-Roig C, Fridlender Z G, Glogauer M, Scapini P. Neutrophil Diversity in Health and Disease. Trends Immunol. 2019; 40(7):565-83. Epub 20190531. doi: 10.1016/j.it.2019.04.012. PubMed PMID: 31160207; PMCID: PMC7185435.
45. Zhou Y, Guo W, Zhu Z, Hu Y, Wang Y, Zhang X, Wang W, Du N, Song T, Yang K, Guan Z, Wang Y, Guo A. Macrophage migration inhibitory factor facilitates production of CCL5 in astrocytes following rat spinal cord injury. J Neuroinflammation. 2018; 15(1):253. Epub 20180904. doi: 10.1186/s12974-018-1297-z. PubMed PMID: 30180853; PMCID: PMC6122456.
46. Liddelow S A, Guttenplan K A, Clarke L E, Bennett F C, Bohlen C J, Schirmer L, Bennett M L, Munch A E, Chung W S, Peterson T C, Wilton D K, Frouin A, Napier B A, Panicker N, Kumar M, Buckwalter M S, Rowitch D H, Dawson V L, Dawson T M, Stevens B, Barres B A. Neurotoxic reactive astrocytes are induced by activated microglia. Nature. 2017; 541(7638):481-7. Epub 20170118. doi: 10.1038/nature21029. PubMed PMID: 28099414; PMCID: PMC5404890.
47. Ito M, Komai K, Mise-Omata S, Iizuka-Koga M, Noguchi Y, Kondo T, Sakai R, Matsuo K, Nakayama T, Yoshie O, Nakatsukasa H, Chikuma S, Shichita T, Yoshimura A. Brain regulatory T cells suppress astrogliosis and potentiate neurological recovery. Nature. 2019; 565(7738):246-50. Epub 20190102. doi: 10.1038/s41586-018-0824-5. PubMed PMID: 30602786.
48. Liao W, Lin J X, Leonard W J. IL-2 family cytokines: new insights into the complex roles of IL-2 as a broad regulator of T helper cell differentiation. Curr Opin Immunol. 2011; 23(5):598-604. Epub 20110831. doi: 10.1016/j.coi.2011.08.003. PubMed PMID: 21889323; PMCID: PMC3405730.
49. Sakaguchi S, Sakaguchi N, Asano M, Itoh M, Toda M. Immunologic self-tolerance maintained by activated T cells expressing IL-2 receptor alpha-chains (CD25). Breakdown of a single mechanism of self-tolerance causes various autoimmune diseases. J Immunol. 1995; 155(3):1151-64. PubMed PMID: 7636184.
50. Drerup J M, Deng Y, Pandeswara S L, Padron A S, Reyes R M, Zhang X, Mendez J, Liu A, Clark C A, Chen W, Conejo-Garcia J R, Hurez V, Gupta H, Curiel T J. CD122-Selective IL2 Complexes Reduce Immunosuppression, Promote Treg Fragility, and Sensitize Tumor Response to P D-L1 Blockade. Cancer Res. 2020; 80(22):5063-75. Epub 20200918. doi: 10.1158/0008-5472.CAN-20-0002. PubMed PMID: 32948605; PMCID: PMC7669742.
51. Ross S H, Cantrell D A. Signaling and Function of Interleukin-2 in T Lymphocytes. Annu Rev Immunol. 2018; 36:411-33. doi: 10.1146/annurev-immunol-042617-053352. PubMed PMID: 29677473; PMCID: PMC6472684.
52. Willerford D M, Chen J, Ferry J A, Davidson L, Ma A, Alt F W. Interleukin-2 receptor alpha chain regulates the size and content of the peripheral lymphoid compartment. Immunity. 1995; 3(4):521-30. doi: 10.1016/1074-7613(95)90180-9. PubMed PMID: 7584142.
53. Sadlack B, Merz H, Schorle H, Schimpl A, Feller A C, Horak I. Ulcerative colitis-like disease in mice with a disrupted interleukin-2 gene. Cell. 1993; 75(2):253-61. doi: 10.1016/0092-8674(93)80067-o. PubMed PMID: 8402910.
54. Evaniew N, Noonan V K, Fallah N, Kwon B K, Rivers C S, Ahn H, Bailey C S, Christie S D, Fourney D R, Hurlbert R J, Linassi A G, Fehlings M G, Dvorak M F, Network R. Methylprednisolone for the Treatment of Patients with Acute Spinal Cord Injuries: A Propensity Score-Matched Cohort Study from a Canadian Multi-Center Spinal Cord Injury Registry. J Neurotrauma. 2015; 32(21):1674-83. Epub 20150717. doi: 10.1089/neu.2015.3963. PubMed PMID: 26065706; PMCID: PMC4638202.
55. Cohen T S, Takahashi V, Bonnell J, Tovchigrechko A, Chaerkady R, Yu W, Jones-Nelson O, Lee Y, Raja R, Hess S, Stover C K, Worthington J J, Travis M A, Sellman B R. Staphylococcus aureus drives expansion of low-density neutrophils in diabetic mice. J Clin Invest. 2019; 129(5):2133-44. Epub 20190415. doi: 10.1172/JC1126938. PubMed PMID: 30985291; PMCID: PMC6486344.
56. Ward N C, Yu A, Moro A, Ban Y, Chen X, Hsiung S, Keegan J, Arbanas J M, Loubeau M, Thankappan A, Yamniuk A P, Davis J H, Struthers M, Malek T R. IL-2/CD25: A Long-Acting Fusion Protein That Promotes Immune Tolerance by Selectively Targeting the IL-2 Receptor on Regulatory T Cells. J Immunol. 2018; 201(9):2579-92. Epub 20181003. doi: 10.4049/jimmunol.1800907. PubMed PMID: 30282751; PMCID: PMC6200646.
57. Lotze M T, Frana L W, Sharrow S O, Robb R J, Rosenberg S A. In vivo administration of purified human interleukin 2. I. Half-life and immunologic effects of the Jurkat cell line-derived interleukin 2. J Immunol. 1985; 134(1):157-66. PubMed PMID: 3871099.
58. Konrad M W, Hemstreet G, Hersh E M, Mansell P W, Mertelsmann R, Kolitz J E, Bradley E C. Pharmacokinetics of recombinant interleukin 2 in humans. Cancer Res. 1990; 50(7):2009-17. PubMed PMID: 2317789.
59. Cengiz S L, Kalkan E, Bayir A, Ilik K, Basefer A. Timing of thoracolomber spine stabilization in trauma patients; impact on neurological outcome and clinical course. A real prospective (rct) randomized controlled study. Arch Orthop Trauma Surg. 2008; 128(9):959-66. Epub 20071127. doi: 10.1007/s00402-007-0518-1. PubMed PMID: 18040702.
60. Ter Wengel P V, Feller R E, Stadhouder A, Verbaan D, Oner F C, Goslings J C, Vandertop W P. Timing of surgery in traumatic spinal cord injury: a national, multidisciplinary survey. Eur Spine J. 2018; 27(8):1831-8. Epub 20180323. doi: 10.1007/s00586-018-5551-y. PubMed PMID: 29572739.
61. Pardi N, Hogan M J, Porter F W, Weissman D. mRNA vaccines—a new era in vaccinology. Nat Rev Drug Discov. 2018; 17(4):261-79. Epub 20180112. doi: 10.1038/nrd.2017.243. PubMed PMID: 29326426; PMCID: PMC5906799.
62. Thomas C E, Ehrhardt A, Kay M A. Progress and problems with the use of viral vectors for gene therapy. Nat Rev Genet. 2003; 4(5):346-58. doi: 10.1038/nrg1066. PubMed PMID: 12728277.
63. Matsuura S, Ono H, Kawasaki S, Kuang Y, Fujita Y, Saito H. Synthetic RNA-based logic computation in mammalian cells. Nat Commun. 2018; 9(1):4847. Epub 20181119. doi: 10.1038/s41467-018-07181-2. PubMed PMID: 30451868; PMCID: PMC6242901.
64. Deyle D R, Russell D W. Adeno-associated virus vector integration. Curr Opin Mol Ther. 2009; 11(4):442-7. PubMed PMID: 19649989; PMCID: PMC2929125.
65. Yamamoto A, Kormann M, Rosenecker J, Rudolph C. Current prospects for mRNA gene delivery. Eur J Pharm Biopharm. 2009; 71(3):484-9. Epub 20081010. doi: 10.1016/j.ejpb.2008.09.016. PubMed PMID: 18948192.
66. Chen C Y, Tran D M, Cavedon A, Cai X, Rajendmn R, Lyle M J, Martini P G V, Miao C H. Treatment of Hemophilia A Using Factor VIII Messenger RNA Lipid Nanoparticles. Mol Ther Nucleic Acids. 2020; 20:534-44. Epub 20200407. doi: 10.1016/j.omtn.2020.03.015. PubMed PMID: 32330871; PMCID: PMC7178004.
67. Verbeke R, Lentacker I, De Smedt S C, Dewitte H. The dawn of mRNA vaccines: The COVID-19 case. J Control Release. 2021; 333:511-20. Epub 20210330. doi: 10.1016/j.jconrel.2021.03.043. PubMed PMID: 33798667; PMCID: PMC8008785.
68. Abbasi J. COVID-19 and mRNA Vaccines-First Large Test for a New Approach. JAMA. 2020; 324(12):1125-7. doi: 10.1001/jama.2020.16866. PubMed PMID: 32880613.
69. Kowalski P S, Rudra A, Miao L, Anderson D G. Delivering the Messenger: Advances in Technologies for Therapeutic mRNA Delivery. Mol Ther. 2019; 27(4):710-28. Epub 20190219. doi: 10.1016/j.ymthe.2019.02.012. PubMed PMID: 30846391; PMCID: PMC6453548.
70. Rohner E, Yang R, Foo K S, Goedel A, Chien K R. Unlocking the promise of mRNA therapeutics. Nat Biotechnol. 2022; 40(11):1586-600. Epub 20221103. doi: 10.1038/s41587-022-01491-z. PubMed PMID: 36329321.
71. Han X, Gong N, Xue L, Billingsley M M, El-Mayta R, Shepherd S J, Alameh M G, Weissman D, Mitchell M J. Ligand-tethered lipid nanoparticles for targeted RNA delivery to treat liver fibrosis. Nat Commun. 2023; 14(1):75. Epub 20230117. doi: 10.1038/s41467-022-35637-z. PubMed PMID: 36650129; PMCID: PMC9845313.
72. LoPresti S T, Arral M L, Chaudhary N, Whitehead K A. The replacement of helper lipids with charged alternatives in lipid nanoparticles facilitates targeted mRNA delivery to the spleen and lungs. J Control Release. 2022; 345:819-31. Epub 20220326. doi: 10.1016/j.jconrel.2022.03.046. PubMed PMID: 35346768; PMCID: PMC9447088.
73. Wang S, Wang H, Drabek A, Smith W S, Liang F, Huang Z R. Unleashing the Potential: Designing Antibody-Targeted Lipid Nanoparticles for Industrial Applications with CMC Considerations and Clinical Outlook. Mol Pharm. 2024; 21(1):4-17. Epub 20231220. doi: 10.1021/acs.molpharmaceut.3c00735. PubMed PMID: 38117251.
74. Su K, Shi L, Sheng T, Yan X, Lin L, Meng C, Wu S, Chen Y, Zhang Y, Wang C, Wang Z, Qiu J, Zhao J, Xu T, Ping Y, Gu Z, Liu S. Reformulating lipid nanoparticles for organ-targeted mRNA accumulation and translation. Nat Commun. 2024; 15(1):5659. Epub 20240705. doi: 10.1038/s41467-024-50093-7. PubMed PMID: 38969646; PMCID: PMC11226454.
75. Saito H, Kobayashi T, Hara T, Fujita Y, Hayashi K, Furushima R, Inoue T. Synthetic translational regulation by an L7Ae-kink-turn RNP switch. Nat Chem Biol. 2010; 6(1):71-8. Epub 20091213. doi: 10.1038/nchembio.273. PubMed PMID: 20016495.
76. Magadum A, Kurian A A, Chepurko E, Sassi Y, Hajjar R J, Zangi L. Specific Modified mRNA Translation System. Circulation. 2020; 142(25):2485-8. Epub 20201221. doi: 10.1161/CIRCULATIONAHA.120.047211. PubMed PMID: 33347328; PMCID: PMC7768930.
77. Koss K M, Son T, Li C, Hao Y, Cao J, Churchward M A, Zhang Z J, Wertheim J A, Derda R, Todd K G. Toward discovering a novel family of peptides targeting neuroinflammatory states of brain microglia and astrocytes. J Neurochem. 2024; 168(10):3386-414. Epub 20230614. doi: 10.1111/jnc.15840. PubMed PMID: 37171455; PMCID: PMC10640667.
78. Benita B A, Koss K M. Peptide discovery across the spectrum of neuroinflammation; microglia and astrocyte phenotypical targeting, mediation, and mechanistic understanding. Front Mol Neurosci. 2024; 17:1443985. Epub 20241120. doi: 10.3389/fnmol.2024.1443985. PubMed PMID: 39634607; PMCID: PMC11616451.
79. Wang Q, Cheng S, Qin F, Fu A, Fu C. Application progress of RVG peptides to facilitate the delivery of therapeutic agents into the central nervous system. RSC Adv. 2021; 11(15):8505-15. Epub 20210224. doi: 10.1039/dira00550b. PubMed PMID: 35423368; PMCID: PMC8695342.
80. Brugnano J L, Chan B K, Seal B L, Panitch A. Cell-penetrating peptides can confer biological function: regulation of inflammatory cytokines in human monocytes by MK2 inhibitor peptides. J Control Release. 2011; 155(2):128-33. Epub 20110512. doi: 10.1016/j.jconrel.2011.05.007. PubMed PMID: 21600941.
81. He Z, Zang H, Zhu L, Huang K, Yi T, Zhang S, Cheng S. An anti-inflammatory peptide and brain-derived neurotrophic factor-modified hyaluronan-methylcellulose hydrogel promotes nerve regeneration in rats with spinal cord injury. Int J Nanomedicine. 2019; 14:721-32. Epub 20190118. doi: 10.2147/IJN.S187854. PubMed PMID: 30705588; PMCID: PMC6342221.
82. Alameh M G, Tombacz I, Bettini E, Lederer K, Sittplangkoon C, Wilmore J R, Gaudette B T, Soliman O Y, Pine M, Hicks P, Manzoni T B, Knox J J, Johnson J L, Laczko D, Muramatsu H, Davis B, Meng W, Rosenfeld A M, Strohmeier S, Lin P J C, Mui B L, Tam Y K, Kariko K, Jacquet A, Krammer F, Bates P, Cancro M P, Weissman D, Luning Prak E T, Allman D, Locci M, Pardi N. Lipid nanoparticles enhance the efficacy of mRNA and protein subunit vaccines by inducing robust T follicular helper cell and humoral responses. Immunity. 2021; 54(12):2877-92 e7. Epub 20211104. doi: 10.1016/j.immuni.2021.11.001. PubMed PMID: 34852217; PMCID: PMC8566475.
83. Sharma P, Hoorn D, Aitha A, Breier D, Peer D. The immunostimulatory nature of mRNA lipid nanoparticles. Adv Drug Deliv Rev. 2024; 205:115175. Epub 20240111. doi: 10.1016/j.addr.2023.115175. PubMed PMID: 38218350.
84. Jovicic A, Roshan R, Moisoi N, Pradervand S, Moser R, Pillai B, Luthi-Carter R. Comprehensive expression analyses of neural cell-type-specific miRNAs identify new determinants of the specification and maintenance of neuronal phenotypes. J Neurosci. 2013; 33(12):5127-37. doi: 10.1523/JNEUROSCI.0600-12.2013. PubMed PMID: 23516279; PMCID: PMC6705001.
85. Park J, Zhang Y, Saito E, Gurczynski S J, Moore B B, Cummings B J, Anderson A J, Shea L D. Intravascular innate immune cells reprogrammed via intravenous nanoparticles to promote functional recovery after spinal cord injury. Proc Natl Acad Sci USA. 2019; 116(30):14947-54. Epub 20190708. doi: 10.1073/pnas.1820276116. PubMed PMID: 31285339; PMCID: PMC6660718.
86. Gal L, Bellak T, Marton A, Fekecs Z, Weissman D, Torok D, Biju R, Vizler C, Kristof R, Beattie M B, Lin P J C, Pardi N, Nogradi A, Pajer K. Restoration of Motor Function through Delayed Intraspinal Delivery of Human IL-10-Encoding Nucleoside-Modified mRNA after Spinal Cord Injury. Research (Wash D C). 2023; 6:0056. Epub 20230309. doi: 10.34133/research.0056. PubMed PMID: 36930811; PMCID: PMC10013810.
87. Ahuja C S, Wilson J R, Nori S, Kotter M R N, Druschel C, Curt A, Fehlings M G. Traumatic spinal cord injury. Nat Rev Dis Primers. 2017; 3:17018. Epub 20170427. doi: 10.1038/nrdp.2017.18. PubMed PMID: 28447605.
88. Lykhopiy V, Malviya V, Humblet-Baron S, Schlenner S M. “IL-2 immunotherapy for targeting regulatory T cells in autoimmunity”. Genes Immun. 2023; 24(5):248-62. Epub 20230923. doi: 10.1038/s41435-023-00221-y. PubMed PMID: 37741949; PMCID: PMC10575774.
89. Gao W, Kim M W, Dykstra T, Du S, Boskovic P, Lichti C F, Ruiz-Cardozo M A, Gu X, Weizman Shapira T, Rustenhoven J, Molina C, Smirnov I, Merbl Y, Ray W Z, Kipnis J. Engineered T cell therapy for central nervous system injury. Nature. 2024; 634(8034):693-701. Epub 20240904. doi: 10.1038/s41586-024-07906-y. PubMed PMID: 39232158.
90. Ransohoff R M. Spinal cord injury: T cells to the rescue? Trends Immunol. 2024; 45(11):849-50. Epub 20241011. doi: 10.1016/j.it.2024.10.001. PubMed PMID: 39395883.
91. Zhang C, Li Y, Yu Y, Li Z, Xu X, Talifu Z, Liu W, Yang D, Gao F, Wei S, Zhang L, Gong H, Peng R, Du L, Li J. Impact of inflammation and Treg cell regulation on neuropathic pain in spinal cord injury: mechanisms and therapeutic prospects. Front Immunol. 2024; 15:1334828. Epub 20240129. doi: 10.3389/fimmu.2024.1334828. PubMed PMID: 38348031; PMCID: PMC10859493.
92. Chen H, Peng H, Wang P C, Zou T, Feng X M, Wan B W. Role of regulatory T cells in spinal cord injury. Eur J Med Res. 2023; 28(1):163. Epub 20230509. doi: 10.1186/s40001-023-01122-6. PubMed PMID: 37161548; PMCID: PMC10169350.
93. Olson K E, Mosley R L, Gendelman H E. The potential for treg-enhancing therapies in nervous system pathologies. Clin Exp Immunol. 2023; 211(2):108-21. doi: 10.1093/cei/uxac084. PubMed PMID: 36041453; PMCID: PMC10019130.
94. Grasshoff H, Comduhr S, Monne L R, Muller A, Lamprecht P, Riemekasten G, Humrich J Y. Low-Dose IL-2 Therapy in Autoimmune and Rheumatic Diseases. Front Immunol. 2021; 12:648408. Epub 20210401. doi: 10.3389/fimmu.2021.648408. PubMed PMID: 33868284; PMCID: PMC8047324.
95. Basso D M, Beattie M S, Bresnahan J C. Graded histological and locomotor outcomes after spinal cord contusion using the NYU weight-drop device versus transection. Exp Neurol. 1996; 139(2):244-56. doi: 10.1006/exnr.1996.0098. PubMed PMID: 8654527.
96. Mukhamedshina Y O, Akhmetzyanova E R, Martynova E V, Khaiboullina S F, Galieva L R, Rizvanov A A. Systemic and Local Cytokine Profile following Spinal Cord Injury in Rats: A Multiplex Analysis. Front Neurol. 2017; 8:581. Epub 20171031. doi: 10.3389/fneur.2017.00581. PubMed PMID: 29163344; PMCID: PMC5671564.
97. Liu C, Hong T, Shao M, Chen Z, Wang C. Melatonin synergized with cyclosporine A improves cardiac allograft survival by suppressing inflammation and apoptosis. Mol Med Rep. 2014; 10(3):1323-8. Epub 20140715. doi: 10.3892/mmr.2014.2382. PubMed PMID: 25059226.
98. Popovich P G, Homer P J, Mullin B B, Stokes B T. A quantitative spatial analysis of the blood-spinal cord barrier. I. Permeability changes after experimental spinal contusion injury. Exp Neurol. 1996; 142(2):258-75. doi: 10.1006/exnr.1996.0196. PubMed PMID: 8934558.
99. Roolfs L, Hubertus V, Spinnen J, Shopperly L K, Fehlings M G, Vajkoczy P. Therapeutic Approaches Targeting Vascular Repair After Experimental Spinal Cord Injury: A Systematic Review of the Literature. Neurospine. 2022; 19(4):961-75. Epub 20221231. doi: 10.14245/ns.2244624.312. PubMed PMID: 36597633; PMCID: PMC9816606.
100. Cao Y, Wu T, Yuan Z, Li D, Ni S, Hu J, Lu H. Three-dimensional imaging of microvasculature in the rat spinal cord following injury. Sci Rep. 2015; 5:12643. Epub 20150729. doi: 10.1038/srep12643. PubMed PMID: 26220842; PMCID: PMC4518284.
101. Ni H, Hatit M Z C, Zhao K, Loughrey D, Lokugamage M P, Peck H E, Cid A D, Muralidharan A, Kim Y, Santangelo P J, Dahlman J E. Piperazine-derived lipid nanoparticles deliver mRNA to immune cells in vivo. Nat Commun. 2022; 13(1):4766. Epub 20220815. doi: 10.1038/s41467-022-32281-5. PubMed PMID: 35970837; PMCID: PMC9376583.
102. Pardi N, Tuyishime S, Muramatsu H, Kariko K, Mui B L, Tam Y K, Madden T D, Hope M J, Weissman D. Expression kinetics of nucleoside-modified mRNA delivered in lipid nanoparticles to mice by various routes. J Control Release. 2015; 217:345-51. Epub 20150808. doi: 10.1016/j.jconrel.2015.08.007. PubMed PMID: 26264835; PMCID: PMC4624045.
103. Gurtner G C, Werner S, Barrandon Y, Longaker M T. Wound repair and regeneration. Nature. 2008; 453(7193):314-21. doi: 10.1038/nature07039. PubMed PMID: 18480812.
104. Kigerl K A, Gensel J C, Ankeny D P, Alexander J K, Donnelly D J, Popovich P G. Identification of two distinct macrophage subsets with divergent effects causing either neurotoxicity or regeneration in the injured mouse spinal cord. J Neurosci. 2009; 29(43):13435-44. doi: 10.1523/JNEUROSCI.3257-09.2009. PubMed PMID: 19864556; PMCID: PMC2788152.
105. Hellenbrand D J, Reichl K A, Travis B J, Filipp M E, Khalil A S, Pulito D J, Gavigan A V, Maginot E R, Arnold M T, Adler A G, Murphy W L, Hanna A S. Sustained interleukin-10 delivery reduces inflammation and improves motor function after spinal cord injury. J Neuroinflammation. 2019; 16(1):93. Epub 20190430. doi: 10.1186/s12974-019-1479-3. PubMed PMID: 31039819; PMCID: PMC6489327.
106. Emam M, Canovas A, Islas-Trejo A D, Fonseca P A S, Medrano J F, Mallard B. Transcriptomic Profiles of Monocyte-Derived Macrophages in Response to Escherichia coli is Associated with the Host Genetics. Sci Rep. 2020; 10(1):271. Epub 20200114. doi: 10.1038/s41598-019-57089-0. PubMed PMID: 31937813; PMCID: PMC6959288.
107. Noristani H N, Gerber Y N, Sabourin J C, Le Corre M, Lonjon N, Mestre-Frances N, Hirbec H E, Perrin F E. RNA-Seq Analysis of Microglia Reveals Time-Dependent Activation of Specific Genetic Programs following Spinal Cord Injury. Front Mol Neurosci. 2017; 10:90. Epub 20170403. doi: 10.3389/fnmol.2017.00090. PubMed PMID: 28420963; PMCID: PMC5376598.
108. Li Y, Lei Z, Ritzel R M, He J, Liu S, Zhang L, Wu J. Ablation of the Integrin CD11b Mac-1 Limits Deleterious Responses to Traumatic Spinal Cord Injury and Improves Functional Recovery in Mice. Cells. 2024; 13(18). Epub 20240920. doi: 10.3390/cells13181584. PubMed PMID: 39329765; PMCID: PMC11430243.
109. Xie X, Shi Q, Wu P, Zhang X, Kambara H, Su J, Yu H, Park S Y, Guo R, Ren Q, Zhang S, Xu Y, Silberstein L E, Cheng T, Ma F, Li C, Luo H R. Single-cell transcriptome profiling reveals neutrophil heterogeneity in homeostasis and infection. Nat Immunol. 2020; 21(9):1119-33. Epub 20200727. doi: 10.1038/s41590-020-0736-z. PubMed PMID: 32719519; PMCID: PMC7442692.
110. Basso D M, Beattie M S, Bresnahan J C. A sensitive and reliable locomotor rating scale for open field testing in rats. J Neurotrauma. 1995; 12(1):1-21. doi: 10.1089/neu.1995.12.1. PubMed PMID: 7783230.
111. Metz G A, Whishaw I Q. The ladder rung walking task: a scoring system and its practical application. J Vis Exp. 2009(28). Epub 20090612. doi: 10.3791/1204. PubMed PMID: 19525918; PMCID: PMC2796662.
112. Deuis J R, Dvorakova L S, Vetter I. Methods Used to Evaluate Pain Behaviors in Rodents. Front Mol Neurosci. 2017; 10:284. Epub 20170906. doi: 10.3389/fnmol.2017.00284. PubMed PMID: 28932184; PMCID: PMC5592204.
113. Hargreaves K, Dubner R, Brown F, Flores C, Joris J. A new and sensitive method for measuring thermal nociception in cutaneous hyperalgesia. Pain. 1988; 32(1):77-88. doi: 10.1016/0304-3959(88)90026-7. PubMed PMID: 3340425.
114. Hellenbrand D J, Kaeppler K E, Hwang E, Ehlers M E, Toigo R D, Giesler J D, Vassar-Olsen E R, Hanna A. Basic techniques for long distance axon tracing in the spinal cord. Microsc Res Tech. 2013; 76(12):1240-9. Epub 20130910. doi: 10.1002/jemt.22291. PubMed PMID: 24022846.
115. Yang E, Nucifora P G, Melhem E R. Diffusion M R imaging: basic principles. Neuroimaging Clin N Am. 2011; 21(1):1-25, vii. doi: 10.1016/j.nic.2011.02.001. PubMed PMID: 21477749.
116. Choudhri A F, Chin E M, Blitz A M, Gandhi D. Diffusion tensor imaging of cerebral white matter: technique, anatomy, and pathologic patterns. Radiol Clin North Am. 2014; 52(2):413-25. doi: 10.1016/j.rcl.2013.11.005. PubMed PMID: 24582347.
117. Vedantam A, Jirjis M B, Schmit B D, Wang M C, Ulmer J L, Kurpad S N. Diffusion tensor imaging of the spinal cord: insights from animal and human studies. Neurosurgery. 2014; 74(1):1-8; discussion; quiz doi: 10.1227/NEU.0000000000000171. PubMed PMID: 24064483; PMCID: PMC4986512.
118. Ahmed R U, Alam M, Zheng Y P. Experimental spinal cord injury and behavioral tests in laboratory rats. Heliyon. 2019; 5(3):e01324. Epub 20190308. doi: 10.1016/j.heliyon.2019.e01324. PubMed PMID: 30906898; PMCID: PMC6411514.

What is claimed:

1. A lipid nanoparticle comprising a therapeutic agent and an astrocyte targeting peptide, wherein the astrocyte targeting peptide is on the surface of the lipid nanoparticle.

2. The lipid nanoparticle of claim 1, wherein the lipid nanoparticle comprises an ionizable lipid, a structural lipid, a sterol, and a pegylated lipid.

3. The lipid nanoparticle of claim 2, wherein the molar ratios of the ionizable lipid, the structural lipid, the sterol, and the pegylated lipid are 50:10:38.5:1.5.

4. The lipid nanoparticle of claim 2, wherein:
a) the ionizable lipid is SM-102;
b) the structural lipid is 1,2-distearoyl-sn-glycero-3-PC (1,2-DSPC);
c) the sterol is cholesterol; and/or
d) the pegylated lipid is selected from a group consisting of 1,2-dimyristoyl-rac-glycero-3-methoxypolyethylene glycol-2000 (DMG-PEG2000), 1,2-distearoyl-sn-glycero-3-phosphoethanolamine-N-[maleimide(polyethylene glycol)-2000], and combinations thereof.

5. The lipid nanoparticle of claim 1, wherein the astrocyte targeting peptide is selected from a group consisting of SEQ ID NO: 1 (AS1-N), SEQ ID NO: 2 (AS1-C), SEQ ID NO: 5 (A-21-1-N), SEQ ID NO: 7 (A+C1-11-N), SEQ ID NO: 8 (A+C1-11-C), SEQ ID NO: 9 (A+C4-9-N), SEQ ID NO: 10 (A+C4-9-C), SEQ ID NO: 12 (ADC6-5-C), SEQ ID NO: 13 (AC2-6-N), and SEQ ID NO: 16 (A+C5-1-C).

6. The lipid nanoparticle of claim 1, wherein the astrocyte targeting peptide is linked to the lipid nanoparticle surface via a pegylated lipid.

7. The lipid nanoparticle of claim 6, wherein the astrocyte targeting peptide comprises a thiol group and the pegylated lipid comprises a maleimide group, and wherein the thiol group is linked to the maleimide group via a thiosuccinimide bond.

8. The lipid nanoparticle of claim 1, wherein the therapeutic agent is a therapeutic mRNA that encodes IL-2, IL-4, IL-10, or IL-13.

9. The lipid nanoparticle of claim 1, wherein the lipid nanoparticle further comprises an anti-inflammatory peptide and optionally wherein the anti-inflammatory peptide comprises a thiol group and the pegylated lipid comprises a maleimide group, and wherein the thiol group is linked to the maleimide group via a thiosuccinimide bond.

10. The lipid nanoparticle of claim 9, wherein the molar ratio of anti-inflammatory peptide to the astrocyte targeting peptide is between 0.1:1 and 1:1.

11. A suppressor mRNA comprising a nucleotide sequence encoding a repressor protein and comprises at least one copy of an astrocyte-expressed miRNA target site in a 5′ untranslated region (UTR) and/or a 3′ UTR of the suppressor mRNA.

12. The suppressor mRNA of claim 11, wherein the at least one copy of the astrocyte-expressed miRNA target site comprises at least one copy of a first astrocyte-expressed miRNA target site and at least one copy of a second astrocyte-expressed miRNA target site.

13. The suppressor mRNA of claim 12, wherein:
a) the first astrocyte-expressed miRNA target site is complementary to miR-449a and the second astrocyte-expressed miRNA target site is complementary to miR-143; or
b) the first astrocyte-expressed miRNA target site is complementary to miR-410 and the second astrocyte-expressed miRNA target site is complementary to miR-135a.

14. The suppressor mRNA of claim 13, wherein the suppressor mRNA comprises SEQ ID NO: 18, SEQ ID NO: 19, or SEQ ID NO: 20.

15. A composition comprising the suppressor mRNA of claim 11 and a therapeutic mRNA, wherein the therapeutic mRNA comprises a repressor binding domain in the 5′ UTR and a nucleotide sequence encoding a therapeutic or reporter protein.

16. A lipid nanoparticle comprising the composition of claim 15.

17. A method of treating a central nervous system (CNS) injury in a subject, the method comprising: administering a lipid nanoparticle comprising a therapeutic agent or therapeutic protein to the subject, wherein the lipid nanoparticle is delivered to the CNS injury.

18. The method of claim 17, wherein the lipid nanoparticle comprises a suppressor mRNA and a therapeutic mRNA, wherein the suppressor mRNA comprises a nucleotide sequence encoding a repressor protein and comprises at least one copy of an astrocyte-expressed miRNA target site in a 5′ untranslated region (UTR) and/or a 3′ UTR of the suppressor mRNA and wherein the therapeutic mRNA comprises a repressor binding domain in the 5′ UTR and a nucleotide sequence encoding a therapeutic or reporter protein.

19. The method of claim 17, wherein the lipid nanoparticle is administered to the subject intravenously.

20. The method of claim 17, wherein the lipid nanoparticle is administered within 8 hours of the CNS injury.

21. The method of claim 17, wherein the CNS injury is a spinal cord injury, a traumatic brain injury, or a hemorrhagic stroke.
